(19) United States
(12) Patent Application Publication
(10) Pub. No.: US 2024/0158751 A1
(43) Pub. Date: May 16, 2024

(54) Methods for Storing Hematopoietic Stem Cells

(71) Applicant: Hemanext Inc., Lexington, MA (US)

(72) Inventor: Samuel O. SOWEMIMO-COKER, Dix Hills, NY (US)

(73) Assignee: Hemanext Inc., Lexington, MA (US)

(21) Appl. No.: 18/549,520

(22) PCT Filed: Mar. 7, 2022
(86) PCT No.: PCT/US2022/019140

§ 371 Date: Sep. 7, 2023

Related U.S. Application Data
(60) Provisional application No. 63/158,267, filed on Mar. 8, 2021.

(51) Int. Cl. C12N 5/0789; A61M 1/36

(52) U.S. Cl. CPC C12N 5/0647; A61M 1/3633; A61M 2202/0208; A61M 2202/0437; C12N 2500/02

(57) ABSTRACT

The present disclosure relates to devices and methods for the improved storage and expansion of stem cells.

### CROSS REFERENCE TO RELATED APPLICATION

This application claims the benefit of U.S. Provisional Application No. 63/158,267 filed Mar. 8, 2021, which is hereby incorporated by reference.

### FIELD OF THE INVENTION

The present disclosure relates to methods for the hypoxic storage of stem cells. The present disclosure also relates to methods for the preconditioning of stem cells. The present disclosure further relates to methods for storing stem cells in hypoxic devices. The present disclosure further relates to methods for the hypoxic growth and proliferation of stem cells from human umbilical cord blood.

### BACKGROUND OF THE INVENTION

Stem cells are the foundation of all parts of the human body. This process of stem cell to tissue, organ, and blood cell development is complex and unpredictable. One of the factors involved in this complex process is oxygen.

Studies show that stem cells reside in microenvironments where they are exposed to

different concentrations of oxygen which play significant roles in their biology, maintenance, differentiation and responses to different stress signals. See Abdollahi H, et al., “The role of hypoxia in stem cell differentiation and therapeutics” J Surg Res, 165(1):112-117 (2011); and Drela K, Sarnowska A, et al., Low oxygen atmosphere facilitates proliferation and maintains undifferentiated state of umbilical cord mesenchymal stem cells in an hypoxia inducible factor-dependent manner. Cytotherapy, 16:881-892 (2014).

Under normal physiological conditions, it is thought that HSCs are maintained in a relatively low proliferative, quiescent state, protected from stress, such as accumulation of reactive oxygen species (ROS) and DNA damage, and preventing their depletion due to excessive proliferation. See Mas-Bargues C, et al. “Relevance of Oxygen Concentration in Stem Cell Culture for Regenerative Medicine” Int. J Mol. Sci., 20 (1195):1-27 (2019); Simon, M. C. and Keith, B. “The role of oxygen availability in embryonic development and stem cell function” Nat. Rev. Mol. Cell Biol. 9, 285-296 (2008); Bigarella C L, and Liang R, Ghaffari S. “Stem cells and the impact of ROS signaling” Development, 141:4206-4218 (2014); Lee J, et al., “Pharmacological regulation of oxidative stress in stem cells” Oxidative Medicine and Cellular Longevity. 1-13 (2018). There is now consensus that oxygen serves as both a metabolic substrate and signaling molecule for cells both in vitro and in vivo. See Mohyeldin A, et al. “Oxygen in stem cell” Cell Stem Cell. 7:150-161 (2010).

In certain types of adult stem cells, low oxygen concentration in vitro promotes proliferation and maintenance of a multipotent state. Grayson W L, et al. “Hypoxia enhances proliferation and tissue formation of human mesenchymal stem cells” Biochem Biophys Res Comm. 358:948-953 (2007); and Csete M. “Oxygen in the cultivation of stem cells” Ann NY Acad Sci.1049:1-8 (2005). Conversely, other investigators have demonstrated hypoxia to be a potent stimulus for differentiation into specific cell lines. Koay E J, et al., “Hypoxic chondrogenic differentiation of human embryonic stem cells enhances cartilage protein synthesis and biomechanical functionality” Osteoarthritis and Cartilege. 10:1-7 (2008); and Khan W S, et al., “Hypoxic conditions increase hypoxia-inducible transcription factor 2a and enhance chondrogenesis in stem cells from the infrapatellar fat pad of osteoarthritis patients” Arthritis Research Ther. 9:1-9 (2007). Alternatively, hypoxia can stimulate cytokine production, thereby potentially playing a role in therapeutic angiogenesis. See Thangarajah H, et al. “IFATS Series: Adipose stromal cells adopt a proangiogenic phenotype under the influence of hypoxia” Stem Cells. 27:266-274 (2009); Sadat S, et al. “The cardioprotective effect of mesenchymal stem cells is mediated by IGF-1 and VEGF” Bioch Biophys Res Comm. 363:674-679 (2007); and Rehman J, et al. “Secretion of angiogenic and anti-apoptotic factors by human adipose stromal cells” Circulation. 109:1292-1298 (2004). Participation in angiogenesis by stem cells may occur directly via differentiation of cells that participate in angiogenesis, or indirectly via cytokine production stimulated by hypoxia. See Cao Y, et al. “Human adipose tissue-derived stem cells differentiate into endothelial cells in vitro and improve postnatal neovascularization in vivo” Biochem Biophys Res Comm. 332:370-379 (2005); and Nakagami H, et al. “Adipose tissue-derived stromal cells as a novel option for regenerative cell therapy” J Atheroslcer Thromb. 13:77-81 (2006). When stem cells are cultured at an oxygen level which is not the same as the one offered by the niche microenvironment, the cells undergo a set of alterations, such as oxidative stress, metabolic turnover, reduced proliferation and self-renewal, hampered motility, altered differentiation potential and a stemness potential loss. All these consequences can be avoided if stem cells are cultured at their physiological oxygen levels.

Here we demonstrate devices and methods for keeping stem cells in a hypoxic state during storage and expansion of stem cells. This hypoxic state provides for improved quality of stem cells for transplantation into patients.

### SUMMARY OF THE INVENTION

The present disclosure provides for, and includes, methods for preparing stem cells for transplantation into a patient in need thereof comprising: collecting a blood product containing stem cells into an oxygen absorbing environment comprising an hypoxic collection container comprising an oxygen (O₂) barrier characterized by an oxygen O₂ permeability of less than 0.5 cc of oxygen per square meter per day and an oxygen sorbent; mixing the blood product until the initial partial pressure of oxygen (pO₂) of the blood product is reduced by between 80 and 95%, separating leukocytes and stem cells from the blood product comprising applying the blood product to a filter wherein the stem cells and leukocytes are retained on the filter to prepare a leukocyte and stem cells depleted blood product; eluting the stem cells from the filter with an isotonic media; and transferring the stem cells into a hypoxic storage container comprising an O₂ barrier characterized by an oxygen (O₂) permeability of less than 0.5 cc of oxygen per square meter per day to prepare hypoxic stored stem cells.

The present disclosure also provides for, and includes, methods for preparing stem cells for transfusion comprising: collecting a blood product comprising stem cells; separating leukocytes and stem cells from the blood product; transferring the stem cells into a hypoxic storage container comprising an O₂ barrier characterized by an oxygen (O₂) permeability of less than 0.5 cc of oxygen per square meter per day, and storing the stem cells in a hypoxic environment of less than 3500 Pa for a period of time at a temperature of less than 37° C.

The present disclosure also provides for, and includes, methods for preparing stem cells for transplantation comprising: collecting human umbilical cord blood (HUCB); separating leukocytes and stem cells from the HUCB by applying HUCB to a leukocyte reduction filter and a stem cell retaining filter; eluting the stem cells from the stem cell retaining filter with an isotonic media; and transferring the stem cells into an hypoxic storage container.

The present disclosure also provides for, and includes, kits for processing stem cells for transplantation comprising: an hypoxic collection container; a leukocyte and stem cell recovery filter; a first accessory hypoxic storage container for collecting filtered supernatant; a second accessory hypoxic storage container comprising stem cell maintaining medium; wherein the second accessory hypoxic storage container is in fluid communication with the leukocyte and stem cell recovery filter, wherein the fluid communication comprises less than 1,400 Pascals (Pa) partial pressure of oxygen (pO₂).

The present disclosure also provides for, and includes, methods of preparing stem cells for transplantation comprising: exposing a hypoxic storage container comprising frozen hypoxic stored stem cells to 37° C.; immersing the hypoxic storage bag comprising frozen hypoxic stored stem cells in a water bath less than 42° C. for thawing the frozen hypoxic stored stem cells to generate thawed stem cells; diluting the thawed stem cells with equal volume of hypoxic solution containing 2.5% (wt/vol) human albumin to form diluted stem cells.

The present disclosure also provides for, and includes, methods of preparing stem cells for transplantation comprising: exposing an hypoxic storage bag comprising frozen hypoxic stored stem cells to at least 25° C. to form thawed hypoxic stored stem cells; centrifuging the thawed hypoxic stored stem cells and removing the supernatant; and resuspending the hypoxic stored stem cells in a solution comprising 3% dextran 40, wherein the hypoxic storage bag comprises a hypoxic environment of less than 3500 Pa.

The present disclosure also provides for, and includes, methods for preparing stem cells for transplantation in a subject in need thereof comprising: thawing hypoxic prepared stem cells; expanding the stem cells in an hypoxic stem cell expansion system comprising; and transplanting the expanded stem cells into the subject in need thereof

The present disclosure further provides for, and includes, methods for preparing cells for transplantation comprising collecting a blood product containing stem cells into an oxygen absorbing environment comprising an hypoxic collection container, the hypoxic collection container comprising an oxygen (O₂) barrier characterized by an O₂ permeability of less than 0.5 cc of oxygen per square meter per day and an oxygen sorbent; mixing the blood product until the initial partial pressure of oxygen (pO₂) of the blood product is reduced by between 80 and 95%; separating leukocytes and stem cells from the blood product comprising applying the blood product to a stem cell binding filter to prepare a leukocyte and stem cell depleted blood product; eluting the stem cells from the filter with an isotonic media; and transferring the stem cells into an hypoxic storage container comprising an inner cell compatible bag comprising a material having an oxygen (O₂) permeability of greater than 25 Barrer and forming hypoxic stored stem cells, wherein the stem cells are not exposed to normoxic conditions for greater than one hour between the collecting and the transferring, wherein the normoxic conditions comprise a partial pressure of oxygen of at least about 21,000 pascal.

### BRIEF DESCRIPTION OF THE DRAWINGS

Some aspects of the disclosure are herein described, by way of example only, with reference to the accompanying drawings. With specific reference now to the drawings in detail, it is stressed that the particulars shown are by way of example and are for purposes of illustrative discussion of embodiments of the disclosure. In this regard, the description, taken with the drawings, makes apparent to those skilled in the art how aspects of the disclosure may be practiced.

FIG. 1 is a schematic for collecting and concentrating stem cells using filtration technology, according to an aspect of the present specification. The schematic shows (A) a hypoxic storage bag containing anticoagulated human umbilical cord blood, (B) a hypoxic storage bag containing post-filtered human umbilical cord blood, and (C) a hypoxic storage bag for collecting the recovered stem cells.

FIG. 2 is a schematic for hypoxic stem cell expansion container, as provided in an aspect of the present specification.

The examples set out herein illustrate several embodiments of the invention but should not be construed as limiting the scope of the invention in any manner.

### DETAILED DESCRIPTION

Unless defined otherwise, technical and scientific terms as used herein have the same meaning as commonly understood by one of ordinary skill in the art. One skilled in the art will recognize many methods can be used in the practice of the present disclosure. Indeed, the present disclosure is in no way limited to the methods and materials described. Any references cited herein are incorporated by reference in their entireties. For purposes of the present disclosure, the following terms are defined below.

As used herein the term “about” refers to ±10%. The terms “comprises,” “comprising,” “includes,” “including,” “having,” and their conjugates mean “including but not limited to.”

The term “consisting of” means “including and limited to.”

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular forms “a,” “an,” and “the” include plural references unless the context clearly dictates otherwise. For example, the term “a compound” or “at least one compound” may include a plurality of compounds, including mixtures thereof.

Throughout this application, various embodiments of this disclosure may be presented in a range format. Accordingly, the description of a range should be considered to have specifically disclosed all the possible subranges as well as individual numerical values within that range. This applies regardless of the breadth of the range. As used herein, “between” means the range includes all the possible subranges as well as individual numerical values within that range but not including the external values. For example, “between 1 and 7” does not include the values 1 or 7 and between “0 and 7” does not include the values 0 or 7.

As used herein the term “method” refers to manners, means, techniques, and procedures for accomplishing a given task including, but not limited to, those manners, means, techniques, and procedures either known to or readily developed from known manners, means, techniques, and procedures by practitioners of the chemical, pharmacological, biological, biochemical, and medical arts.

As used herein, the term “bag” refers to collapsible containers prepared from a flexible material and includes pouches, tubes, and gusset bags. In certain aspects, a bag refers to non-collapsible container. As used herein, and included in the present disclosure, the term bag includes folded bags having one, two, three, or more folds and which are sealed or bonded on one, two, three, or more sides. Bags are prepared using a variety of techniques known in the art including bonding of sheets of one or more materials. Methods of bonding materials to form bags are known in the art. See International Publication No. WO 2016/145210. Also included and provided for in the present disclosure are containers prepared by injection and blow molding. Methods to prepare blow molded and injection molded containers are known in the art. See U.S. Pat. Nos. 4,280,859; and 9,096,010.

Preferred types of blow molded or injection molded containers are flexible containers that can be reduced in size for efficient packing and shipping while being capable of expanding to accommodate blood or blood components for reduction of oxygen. They also may be designed to conform to the volume of the blood until they are fully expanded. As used throughout the present disclosure, the bags are a form of collapsible container and the two terms are used interchangeably throughout the present disclosure.

As used herein the term “normoxic” or “normoxic conditions” refer to blood products or an environment having a non-depleted level of oxygen. In an aspect, normoxic or normoxic conditions refer to a partial pressure of oxygen of at least 21,000 Pa. As used herein the term “medio-oxic” or “medio-oxic conditions” refer to blood products or an environment having a non-depleted level of oxygen and minimum oxygen ingress. In aspect, medio-oxic or medio-oxic conditions refer to a partial pressure of oxygen of at least 13,000 Pa. In another aspect, medio-oxic or medio-oxic conditions refer to a partial pressure of oxygen of between 13,000 Pa and 20,000 Pa. As used herein the term “hypoxic” or “hypoxic conditions” refer to blood products or an environment having a depleted level of oxygen. In an aspect, hypoxic or hypoxic conditions refer to a partial pressure of oxygen of less than 12,000 Pa. In another aspect, hypoxic or hypoxic conditions refer to between 400 and 12,000 Pa. In yet another aspect, hypoxic or hypoxic conditions refer to between 2,000 and 10,000 Pa, 3,000 and 10,000 Pa, 4,000 and 10,000 Pa, 2,000 and 12,000 Pa, 3,000 and 12,000 Pa, or 4,000 and 12,000 Pa.

As used herein the terms “blood” and “blood product” refers to peripheral blood, human umbilical cord blood (HUCB), and bone marrow that contain stem cells. The temperature of blood varies with the stage of the collection process, starting at the normal body temperature of 37 ° C. at the time and point of collection, but decreasing rapidly to about 30° C. once removed from the patient's body. A unit of collected blood cools to room temperature in about 6 hours when untreated. In practice, the blood for transfusion is processed within 24 hours and refrigerated at between about 2° C. and 6° C., usually 4 ° C.

As used herein, the term “blood stem cell” or “stem cell” refers to an immature cell

that can develop into all types of blood cells, including red blood cells, white blood cells, and platelets. Blood stem cells are also known as hematopoietic stem cells. Blood stem cells are found in and extracted from a donor's umbilical cord blood, bone marrow and peripheral blood.

Stem cells are undifferentiated or partially differentiated cells with the ability to differentiate into various types of cells and divide indefinitely to produce the same. These daughter cells either become new stem cells (self-renewal) or become specialized cells (differentiation) with a more specific function, such as blood cells, brain cells, heart muscle cells or bone cells. See Morrison S J, et al., Regulatory mechanisms in stem cell biology, Cell, 88: 287-298 (1997) (Morrison 1997); and Reubinoff B E, et al., Embryonic stem cell lines from human blastocysts: somatic differentiation in vitro, Nat. Biotechnol, 18: 399-404 (2000) (Reubinoff 2000) (hereby incorporated by reference in their entireties).

Not to be limited by theory, recent evidence demonstrates that stem cells can be employed to repopulate many, if not all, tissues and restore physiologic and anatomic functionality. Accordingly, stem cells have the potential to be used in treating a wide variety of diseases and injuries, including nervous system trauma, malignancies, genetic diseases, hemoglobinopathies, and immunodeficiency. Generally, stem cells are divided into two types: Embryonic Stem (ES) cells and Adult Stem (AS) cells. Embryonic stem cells are prepared from embryos that are three to five days old. At this stage the embryo is called a blastocyst and has about 150 cells. See Morrison 1997 and Reubinoff 2000. ES cells are understood to be totipotent.

Adult stem cells can rapidly replenish lost cell types through the natural cell death cycle, injury, or disease. Adult stem cells can differentiate into a few cell types (pluripotent or multipotent) or can be limited to one cell type (unipotent).

One class of pluripotent stem cells are hematopoietic stem cells (HSCs) which are responsible for replenishing blood and immune cells. Hematopoietic stem cells (HSCs) are the multipotent stem cells, which are able to give rise to all types of blood cells including myeloid (monocytes, macrophages, neutrophils, basophils, eosinophils, erythrocytes, megakaryocytes/platelets, and dendritic cells) and lymphoid lineages (T cells, B cells, NK cells). Adult stem cells are vastly outnumbered by the progeny cells and terminally differentiated cells that they differentiate into. See McKee C, et al., Advances and challenges in stem cell culture. Colloids and Surf B: Biointerfaces, 159:62-77 (2017) and Bieniasz M, et al. Stem cell general characteristics and sources. MEDtube Science, 2:8-14 (2014) (hereby incorporated by reference in their entireties).

Adult stem cells can be altered to have more of the totipotent properties of embryonic stem cells and are called induced pluripotent cells (iPSCs). The three main donor sources of adult stem cells are the bone marrow, adipose tissue and peripheral blood. Human umbilical cord blood (HUCB) is also a rich source of adult stem cells including hematopoietic stem cells (HSCs), mesenchymal stem cells (MSCs) and other progenitor cells. Hematopoietic stem and progenitor cells (HSPC) as well as some mature cells express an antigen on their surfaces termed CD34 and CD34 is used widely as one identifier of haemopoietic stem cells and has been used as a dose requirement in protocols for stem cell transplantation.

The present disclosure provides for, and includes, hematopoietic stem cells comprising one or more hematopoietic stem cell markers selected from the group consisting of polycomb group RING finger protein 4 (BMI-1), cluster of differentiation 21 (CD21), cluster of differentiation 22 (CD22), cluster of differentiation 34 (CD34), cluster of differentiation 38 (CD38), cluster of differentiation 41 (CD41), cluster of differentiation 44 (CD44), cluster of differentiation 45 (CD45), cluster of differentiation 48 (CD48), cluster of differentiation 90 (CD90; Thy 1), cluster of differentiation 105 (CD105), cluster of differentiation 106 (CD106), cluster of differentiation 117 (CD117; c-kit), cluster of differentiation 127 (CD127), cluster of differentiation 150 (CD150), c-myc, endothelial protein c receptor (EPCR), lymphocyte antigen-6 (Ly6A/E; sca-1), MYB, induced myeloid leukemia cell differentiation protein (Mcl-1), phosphatase and tensin homolog (PTEN), Skp, Cullin, F-box (SCF; kit ligand), single transducer and activator of transcription 5a (STAT5a), single transducer and activator of transcription 5b (STATSb), and vascular endothelial growth factor receptor 2 (VEGFR2). In another aspect, hematopoietic stem cells comprise two or more hematopoietic stem cell markers selected from the group consisting of polycomb group RING finger protein 4 (Bmi-1), cluster of differentiation 21 (CD21), cluster of differentiation 22 (CD22), cluster of differentiation 34 (CD34), cluster of differentiation 38 (CD38), cluster of differentiation 41 (CD41), cluster of differentiation 44 (CD44), cluster of differentiation 45 (CD45), cluster of differentiation 48 (CD48), cluster of differentiation 90 (CD90; Thy 1), cluster of differentiation 105 (CD105), cluster of differentiation 106 (CD106), cluster of differentiation 117 (CD117; c-kit), cluster of differentiation 127 (CD127), cluster of differentiation 150 (CD150), c-myc, endothelial protein c receptor (EPCR), lymphocyte antigen-6 (Ly6A/E; sca-1), MYB, induced myeloid leukemia cell differentiation protein (Mcl-1), phosphatase and tensin homolog (PTEN), Skp, Cullin, F-box (SCF; kit ligand), single transducer and activator of transcription 5a (STAT5a), single transducer and activator of transcription 5b (STAT5b), and vascular endothelial growth factor receptor 2 (VEGFR2).

In an aspect, the present disclosure provides for, and includes, stem cells which are mesenchymal stem cells (MSCs) comprising one or more mesenchymal stem cell markers selected from the group consisting CD44, CD90, CD105, CD106, CD166, and Stro-1. In another aspect, the present disclosure provides for mesenchymal stem cells (MSCs) comprising two or more mesenchymal stem cell markers selected from the group consisting CD44, CD90, CD105, CD106, CD166, and Stro-1. In yet another aspect, mesenchymal stem cells (MSCs) comprising three or more mesenchymal stem cell markers selected from the group consisting CD44, CD90, CD105, CD106, CD166, and Stro-1. In another aspect, hematopoietic stem cells comprise one or more hematopoietic stem cell markers selected from the group selected from the group of polycomb group proteins consisting of Bmi1, Mel18, Rae28, Cbx2, Cbx8, Ring1B, Ezh1, Ezh2, Eed, and Suzl2. See Takamatsu-Ichihara, E. and Kitabayashi, I., “The roles of Polycomb group proteins in hematopoetic stem cells and hematological malignancies” Intern Jour. Of Hematology. 103, 634-642 (2016).

The present disclosure provides for, and includes, a method for preparing stem cells in a hypoxic condition during collection and concentration to improve the quality and proliferative potential of the stem cells. More specifically, the methods of the present disclosure provide for excluding oxygen during the collection and expansion stages.

In an aspect, the present disclosure provides methods for preparing stem cells for transplantation into a patient in need thereof comprising collecting a blood product containing stem cells into an oxygen absorbing environment having a low partial pressure of oxygen, mixing the blood product to reduce oxygen, separating the leukocytes and stem cells from the blood product, and transferring the stem cells into a hypoxic storage container. In an aspect, the stem cells are stored for up to 3 days at room temperature. In another aspect, the stem cells are stored for at least 1 day at room temperature. In another aspect, the stem cells are stored frozen. In another aspect, the stem cells are transplanted into a patient in need of a stem cell transplant. Room temperature is between 20 and 22° C. Room temperature is between 19 and 25° C. Room temperature is at least 20° C. Room temperature is less than 22° C. Room temperature is between 20 and 23° C. Room temperature is between 19 and 22° C. In another aspect of the present disclosure, the stem cells are stored at room temperature for less than 7 days. In another aspect, the stem cells are stored at room temperature for 1 to 7 days. In another aspect, the stem cells are stored at room temperature for at least 1 day, at least 3 days, at least 5 days, and at least 7 days. In another aspect, the stem cells are stored at 4° C. for 1 to 7 days, 1 to 3 days, 1 to 5 days, 1 to 14 days, 2 to 7 days, 2 to 8 days, 4 to 7 days, 5 to 10 days, or 3 to 14 days. In another aspect, the stem cells are stored at 4° C. for less than 14 days, less than 10 days, less than 7 days, less than 5 days, and less than 3 days. In yet another aspect, the stem cells are stored at 4° C. for at least 1 day, at least 3 days, at least 5 days, at least 7 days, and at least 10 days.

The present disclosure provides for, and includes, a method for preparing stem cells for transplantation into a patient in need thereof comprising collecting a blood product containing stem cells into an oxygen absorbing environment comprising an hypoxic collection container comprising an O₂ barrier characterized by an O₂ permeability of less than 0.5 cc of oxygen per square meter per day and an oxygen sorbent, mixing the blood product until the initial partial pressure of oxygen (pO₂) of the blood product is reduced by between 80 and 95%, separating leukocytes and stem cells from said blood product by applying the blood product to a stem cell and leukocyte binding filter to produce a stem cell depleted blood product and a stem cell bound filter at a low partial pressure of oxygen. The stem cells are eluted from the filter with an isotonic media and transferred into a hypoxic storage container comprising an O₂ barrier characterized by an oxygen permeability of less than 0.5 cc of oxygen per square meter per day to prepare hypoxic stem cells. The stem cells can be stored at temperatures below 37° C. under hypoxic conditions to prepare stored hypoxic stem cells.

The present disclosure provides for, and includes, a method for preparing stem cells for transfusion comprising collecting a blood product comprising stem cells, separating leukocytes and stem cells from said blood product, transferring said stem cells into a hypoxic storage container comprising an O₂ barrier characterized by an O₂ permeability of less than 0.5 cc of oxygen per square meter per day, and storing said stem cells in a hypoxic environment of less than for a period of time at a temperature of 37° C. or less. The present disclosure provides for, and includes, a method for preparing stem cells

for transplantation comprising collecting human umbilical cord blood (HUCB), separating leukocytes and stem cells from said HUCB by applying UCB to a leukocyte reduction filter and a stem cell retaining filter, eluting said stem cells from said stem cell retaining filter with an isotonic media; and transferring said stem cells into an hypoxic storage container.

The present disclosure provides for, and includes, a method for preparing stem cells for transplantation comprising collecting human umbilical cord blood (HUCB), separating leukocytes and stem cells from said HUCB by applying UCB to a leukocyte reduction filter and a stem cell retaining filter, eluting said stem cells from said stem cell retaining filter with an isotonic media; and transferring said stem cells into an medio-oxic storage container, wherein the medio-oxic container prevents oxygen ingress.

In an aspect of the present disclosure a method for preparing stem cells for transplantation into a patient in need of stem cell transplant is provided. In another aspect, a patient in need thereof is a person having cancer or immune system disease. In another aspect, a patient in need thereof and in need of stem cell transplantation is a patient having a malignant or nonmalignant of blood or bone marrow. In yet another aspect, a person in need of stem cell transplant is a person having acute lymphoblastic leukemia (ALL), acute myeloid leukemia (AML), aplastic anemia and paroxysmal nocturnal hemoglobinuria (PNH), chronic lymphocytic leukemia (CLL), chronic myelogenous leukemia (CML), Hodgkin lymphoma,

Non-Hodgkin lymphoma, multiple myeloma, myelodyspastic syndrome, Waldenstrom's macroglobulinemia, testicular cancer, congenital disorders of blood production (e.g. sickle cell anemia, thalassemia), Diamond Blackfan anemia (DBA), Shwachman-Diamond syndrome (SDS), dyskeratosis congenita syndromes, or an autoimmune disorder. In another aspect, a person in need of stem cell transplant is a person having acute lymphoblastic leukemia (ALL). In another aspect, a person in need of stem cell transplant is a person having acute myeloid leukemia (AML). In another aspect, a person in need of stem cell transplant is a person having aplastic anemia. In another aspect, a person in need of stem cell transplant is a person having paroxysmal nocturnal hemoglobinuria (PNH). In another aspect, a person in need of stem cell transplant is a person having chronic lymphocytic leukemia (CLL). In another aspect, a person in need of stem cell transplant is a person having chronic myelogenous leukemia (CML). In another aspect, a person in need of stem cell transplant is a person having Hodgkin lymphoma. In another aspect, a person in need of stem cell transplant is a person having Non-Hodgkin lymphoma. In another aspect, a person in need of stem cell transplant is a person having multiple myeloma. In another aspect, a person in need of stem cell transplant is a person having myelodyspastic syndrome. In another aspect, a person in need of stem cell transplant is a person having Waldenstrom's macroglobulinemia. In another aspect, a person in need of stem cell transplant is a person having testicular cancer. In another aspect, a person in need of stem cell transplant is a person having congenital disorders of blood production (e.g. sickle cell anemia, thalassemia). In another aspect, a person in need of stem cell transplant is a person having Diamond Blackfan anemia (DBA). In another aspect, a person in need of stem cell transplant is a person having Shwachman-Diamond syndrome (SDS). In another aspect, a person in need of stem cell transplant is a person having dyskeratosis congenita syndromes. In another aspect, a person in need of stem cell transplant is a person having an autoimmune disorder. In an aspect, the present disclosure provides for, and includes, a volume of stem cells appropriate for transplantation in a patient in need thereof. In another aspect, an appropriate volume for transplantation is between 10 and 50 milliliters per kilogram (mL/kg). In another aspect, an appropriate volume for transplantation is between 20 and 40 mL/kg. In another aspect, an appropriate volume for transplantation is at least 10 mL/kg. In another aspect, an appropriate volume for transplantation is at least 20 mL/kg. In a further aspect, an appropriate volume is 30 mL/kg.

The present disclosure provides for, and includes, a method for collection of a blood product containing stem cells in a hypoxic collection bag. In aspects as provided herein, the hypoxic collection bag contains an anticoagulant to prevent coagulation of the blood or clumping of the stem cells. In an aspect, the anticoagulant is selected from the group consisting of citrate-phosphate dextrose (CPD), citrate—phosphate—dextrose—adenine 1 (CPDA-1), acid citrate dextrose (ACD) and heparin. In another aspect, the anticoagulant is citrate-phosphate dextrose (CPD). In another aspect the anticoagulant is citrate—phosphate— dextrose—adenine 1 (CPDA-1). In another aspect, the anticoagulant is acid citrate dextrose (ACD). In yet another aspect, the anticoagulant is heparin.

In aspects, the blood product containing stem cells collected in a hypoxic collection bag is mixed to increase oxygen depletion from the blood product. In aspects provided herein, it is important to reduce the partial pressure of oxygen of the blood product containing stem cells collected in a hypoxic collection bag as quickly as possible. In an aspect, the blood product is mixed in the oxygen absorbing environment until the initial partial pressure of oxygen (pO₂) of the blood product is reduced by between 80 and 95% after 1 to 3 hours of mixing. In another aspect, the blood product is mixed in the oxygen absorbing environment until the initial partial pressure of oxygen (pO₂) of the blood product is reduced by between 70 and 95%, between 60 and 95%, between 50 and 95%, between 40 and 95%, between 30 and 95%, between 30 and 70%, between 30 and 80% within 3 hours. In yet another aspect, the blood product is mixed in the oxygen absorbing environment until the initial partial pressure of oxygen (pO₂) of the blood product is reduced by at least, 30, 40, 50, 60, 70, 80, 90, or 95% within 3 hours of mixing. In yet another aspect, the blood product is mixed for less than 3 hours in the oxygen absorbing environment until the initial partial pressure of oxygen (pO₂) of the blood product is reduced by at least, 30, 40, 50, 60, 70, 80, 90, or 95%.

In another aspect, the blood product containing stem cells is mixed until the pO₂ is less than 3500 Pascals (Pa; equivalent to 26 mmHg), less than 3000 Pa, less than 2500 Pa, less than 2000 Pa, less than 1500 Pa, or less than 1000 Pa. In aspects, the pO₂ of the blood product containing stem cells is reduced within 3 hours of collection. In another aspect, the blood product is mixed until the pO₂ is between 990 and 3500 Pa, between 990 and 3000 Pa, between 990 and 2500 Pa, between 990 and 2000 Pa, between 990 and 1500 Pa, or between 1000 and 3000 Pa. In an aspect of the present disclosure the blood product is mixed under a pO₂ of between 400 and 2000 Pa, between 500 and 2000 Pa, between 600 and 2000 Pa, between 700 and 2000 Pa, between 800 and 2000 Pa, between 900 and 2000 Pa, between 900 and 2000 Pa, between 1000 and 2000 Pa, between 1200 and 2000 Pa, between 1400 and 2000 Pa, between 1600 and 2000 Pa, between 1800 and 2000 Pa. In another aspect, the blood product is mixed under a pO₂ of between 900 and 3500 Pa or 2000 and 3500 Pa.

In an aspect of the present disclosure, the blood product is mixed for up to 3 hours under a partial pressure of oxygen (pO₂) of between 900 and 3500 Pa. In another aspect, the blood product is mixed for no more than 1 hour and until the initial partial pressure of oxygen (pO₂) of the blood product is reduced by between 80 and 95%. In another aspect, the blood product is mixed for no more than 2 hour and until the initial partial pressure of oxygen (pO₂) of the blood product is reduced by between 80 and 95%. In another aspect, the blood product is mixed for up to 3 hours and until the initial partial pressure of oxygen (pO₂) of the blood product is reduced by between 80 and 95%. In yet another aspect, the blood product is mixed for between 1 and 3 hours until the initial partial pressure of oxygen (pO₂) of the blood product is reduced by between 70 and 95%.

The present disclosure provides various methods for separating stem cells from a blood product under hypoxic conditions. In an aspect of the present disclosure, stem cells are separated from a blood product (i.e., peripheral blood, human umbilical cord blood, or bone marrow) by affinity chromatography with a stem cell retaining filter. In another aspect, stem cells or stem cells and leukocytes are separated from a blood product by filtration with a leukocyte reduction and stem cell retaining filter. Various leukocyte reduction filters are commonly known in the art, including the Haemonetics® leukocyte reduction filter (BPF4 leukocyte removal filter, Haemonetics®, Braintree, MA). In yet another aspect, the leukocyte reduction and stem cell retaining filters are combined into a single filter. In another aspect, the stem cell retaining filter comprises a stem cell specific monoclonal antibody. In another aspect, the stem cell retaining filter comprises an antibody for CD34. In another aspect, the stem cell retaining filter comprises one or more antibodies selected from the group consisting of CD45, CD90, CD3e, CD34, CD49f (Integrin a6) cKit/CD117, Ly6A/E (Sca-1), CD13, CD29,CD36,CD44, CD73, CD105, and CD146. In another aspect, the stem cell retaining filter comprises two or more antibodies selected from the group consisting of CD45,CD90, CD3e, CD34, CD49f (Integrin a6) cKit/CD117, Ly6A/E (Sca-1), CD13, CD29,CD36,CD44, 30 CD73, CD105, and CD146. In a further aspect, the stem cell retaining filter comprises three or more antibodies selected from the group consisting of CD45,CD90, CD3e, CD34, CD49f (Integrin a6) cKit/CD117, Ly6A/E (Sca-1), CD13, CD29,CD36,CD44, CD73, CD105, and CD146. In a further aspect, the stem cell retaining filter comprises four or more antibodies selected from the group consisting of CD45,CD90, CD3e, CD34, CD49f (Integrin a6) cKit/CD117, Ly6A/E (Sca-1), CD13, CD29,CD36,CD44, CD73, CD105, and CD146. The stem cells are concentrated on the filter by binding and eluted from the filter using an isotonic media. In aspects, the isotonic media is an oxygen reduced media having a pO₂ of no greater than 3500 Pa.

In another aspect, stem cells are separated from a blood product by centrifugation of bone marrow aspirate, cord blood, peripheral blood, lipoaspirate, or a mixture thereof. The supernatant is removed, and the stem cells are resuspended in stem cell maintaining solution. An example of a stem cell maintaining solution, is phosphate buffered saline with 3% wt/vol dextran. Another example of a stem cell maintaining solution, is phosphate buffered with 5% human albumin. In an aspect, the stem cells are separated from a hypoxic blood product. In another aspect, the stem cells are separated from a normoxic blood product. In another aspect, a blood product is mixed with a density gradient prior to centrifugation.

In an aspect of the present disclosure the blood product is separated by filtration or centrifugation under reduced oxygen conditions where the pO₂ is maintained between 400 and 2000 Pa, between 500 and 2000 Pa, between 600 and 2000 Pa, between 700 and 2000 Pa, between 800 and 2000 Pa, between 900 and 2000 Pa, between 900 and 2000 Pa, between 1000 and 2000 Pa, between 1200 and 2000 Pa, between 1400 and 2000 Pa, between 1600 and 2000 Pa, between 1800 and 2000 Pa. In another aspect, the blood product is separated under a pO₂ of between 900 and 3500 Pa or 2000 and 3500 Pa.

In an aspect, the blood product being passed through the filter includes red blood cells that have been treated to have an oxygen saturation of less than 20%. In aspects, red blood cell containing blood products having stem cells can be obtained from peripheral blood (e.g., whole blood) or from umbilical cord blood and bone marrow. In another aspect, the red blood cell containing blood products having stem cells (e.g., whole blood) is treated to have an oxygen saturation of less than 20% prior to extraction of stem cells. In another aspect, the blood product being passed through the filter comprises red blood cells having an oxygen saturation of less than 15% after deoxygenation treatment. In another aspect, the blood product being passed through the filter comprises red blood cells having an oxygen saturation of less than 10% after deoxygenation treatment. In another aspect, the blood product being passed through the filter comprises red blood cells having an oxygen saturation of less than 5% after deoxygenation treatment. In yet another aspect, the blood product being passed through the filter comprises red blood cells having an oxygen saturation of between 4 and 25% after deoxygenation treatment. In another aspect, the blood product being passed through the filter comprises red blood cells having an oxygen saturation of between 4 and 20% after deoxygenation treatment. In another aspect, the blood product being passed through the filter comprises red blood cells having an oxygen saturation of between 10 and 20% after deoxygenation treatment. Methods of efficiently reducing oxygen from peripheral blood are provided in U.S. International Publication Nos. WO 2016/145210, published Sep. 15, 2016 and WO 2016/029069, published Oct. 27, 2016.

In an aspect of the present disclosure, the leukocyte filter, stem cell recovery filter, or leukocyte and stem cell recovery filter as well as the solutions are maintained under hypoxic conditions comprising a partial pressure of oxygen of less than 3000 Pa pO₂. In aspects, the cells, devices, and solutions are maintained at a partial pressure of oxygen of less than 2500 Pa pO₂, less than 2000 Pa pO₂, less than 1400 Pa pO₂, or less than 900 Pa pO₂. In another aspect, the leukocyte filter, stem cell recovery filter, or leukocyte and stem cell recovery filter are maintained under hypoxic conditions comprising between 900 and 3000 Pa pO₂, 900 and 2500 Pa pO₂, 900 and 2000 Pa pO₂, 1400 and 2500 Pa pO₂, or between 1300 and 2500 Pa pO₂. In another aspect, the leukocyte filter, stem cell recovery filter, or leukocyte and stem cell recovery filter are maintained under hypoxic conditions comprising a partial pressure of oxygen of less than 3000 Pa pO₂ and a partial pressure of carbon dioxide of less than 6000 Pa. In yet another aspect, the leukocyte filter, stem cell recovery filter, or leukocyte and stem cell recovery filter are maintained under hypoxic conditions comprising a partial pressure of oxygen of as provided in this paragraph and a partial pressure of carbon dioxide of less than 6000 Pa.

The present disclosure provides for, and includes, methods for eluting stem cells concentrated on a stem cell recovery filter. In an aspect, the concentrated stem cells are eluted under hypoxic conditions in an isotonic media. In certain aspects, the isotonic media is an isotonic media. In other aspects, the isotonic media is compatible with tissue culture of stem cells. In some aspects, the isotonic media can be combined with additional components to prepare an isotonic media. In another aspect, the present disclosure provides for eluting concentrated stem cells with an isotonic media. In another aspect, eluting comprises treating the concentrated stem cells with 50 to 200 mL of an isotonic media. In another aspect, eluting comprises treating the concentrated stem cells with at least 50 mL of an isotonic media. In another aspect, eluting comprises treating the concentrated stem cells with at least 100 mL of an isotonic media. In another aspect, eluting comprises treating the concentrated stem cells with at least 150 mL of an isotonic media. In another aspect, eluting comprises treating the concentrated stem cells with 100 mL of an isotonic media.

The present disclosure provides for, and includes, isotonic media characterized by the partial pressure of oxygen. In aspects, the isotonic media is normoxic (e.g., equilibrated with oxygen at ambient pressure). In other aspects, the isotonic media has a reduced partial pressure of oxygen. In aspects, the isotonic media and components have a partial pressure of oxygen below 3500 Pa. In another aspect the isotonic media is deoxygenated isotonic media comprising a pO₂ of less than 3000 Pa, less than 2500 Pa, less than 2000 Pa, less than 1500 Pa, or less than 1000 Pa. In another aspect, the isotonic media is deoxygenated isotonic media comprising a pO₂ of between 990 and 6000 Pa, between 990 and 3500 Pa, between 990 and 3000 Pa, between 990 and 2500 Pa, between 990 and 2000 Pa, between 990 and 1500 Pa, or between 1000 and 3000 Pa. In an aspect of the present disclosure the isotonic media is deoxygenated isotonic media comprising a pO₂ of between 400 and 2000 Pa, between 500 and 2000 Pa, between 600 and 2000 Pa, between 700 and 2000 Pa, between 800 and 2000 Pa, between 900 and 2000 Pa, between 900 and 2000 Pa, between 1000 and 2000 Pa, between 1200 and 2000 Pa, between 1400 and 2000 Pa, between 1600 and 2000 Pa, between 1800 and 2000 Pa. In another aspect, the isotonic media is deoxygenated isotonic media comprising a pO₂ of between 900 and 3000 Pa or 2000 and 3000 Pa. In another aspect, the concentrated stem cells are eluted into a container. In yet another aspect, the concentrated stem cells are eluted directly into a hypoxic storage container.

In an aspect of the present disclosure the stem cells are eluted under a pO₂ of between 400 and 2000 Pa, between 500 and 2000 Pa, between 600 and 2000 Pa, between 700 and 2000 Pa, between 800 and 2000 Pa, between 900 and 2000 Pa, between 900 and 2000 Pa, between 1000 and 2000 Pa, between 1200 and 2000 Pa, between 1400 and 2000 Pa, between 1600 and 2000 Pa, between 1800 and 2000 Pa. In another aspect, the stem cells are eluted under a pO₂ of between 900 and 4000 Pa or 2000 and 4000 Pa.

In an aspect of the present disclosure, stem cells are separated from a blood product and stored under hypoxic conditions. In an aspect, the separated stem cells are transferred into a hypoxic storage container under a pO₂ of between 400 and 2000 Pa, between 500 and 2000 Pa, between 600 and 2000 Pa, between 700 and 2000 Pa, between 800 and 2000 Pa, between 900 and 2000 Pa, between 900 and 2000 Pa, between 1000 and 2000 Pa, between 1200 and 2000 Pa, between 1400 and 2000 Pa, between 1600 and 2000 Pa, between 1800 and 2000 Pa. In another aspect, the stem cells are transferred into a hypoxic storage container under a pO₂ of between 900 and 3500 Pa or 2000 and 3500 Pa.

In an aspect, the stem cell suspension (stem cells with stem cell maintaining solution) is stored in a hypoxic storage bag at room temperature for up to 3 days. In another aspect, the stem cell suspension is diluted with a cryoprotectant and stored at −80° C., or in liquid nitrogen (approximately −195° C.). In an aspect, cryoprotectants for freezing a stem cell suspension are selected from the group consisting of trehalose, mannitol, sucrose, ethylene glycol, dimethyl sulfoxide, dextran, hydroxyethyl starch, glucose, glycerol, polyvinyl pyrrolidone, propylene glycol, polyethylene glycol, 2-methyl-2,4-pentanediol, formamide, glycerol-3phosphate, proline, sorbitol, diethyl glycol, triethylene glycol, and combinations thereof. In another aspect, stem cell suspensions are frozen in a solution comprising trehalose and dextran.

The present disclosure provides for, and includes, collecting a blood product, separating stem cells and further expanding stem cells by culturing under hypoxic conditions over 3 to 4 days and expanding the stem cells between 3 and 14 fold. For example, the present disclosure provides for collecting a blood product containing stem cells into an oxygen absorbing environment, mixing the blood product until the initial partial pressure of oxygen (pO₂) of the blood product is reduced by between 80 and 95%, separating leukocytes and stem cells from the blood product, expanding the stem cells, and transferring stems cells to a hypoxic storage container.

In an aspect of the present disclosure, the separated stem cells are transferred to a hypoxic stem cell expansion container prior to transferring stem cells to a hypoxic storage container. In another aspect, the separated stem cells are transferred to a hypoxic storage container prior to transferring the stem cells to a hypoxic stem cell expansion container. In another aspect, separated stem cells are transferred to a hypoxic expansion container and then transplanted into a patient in need thereof

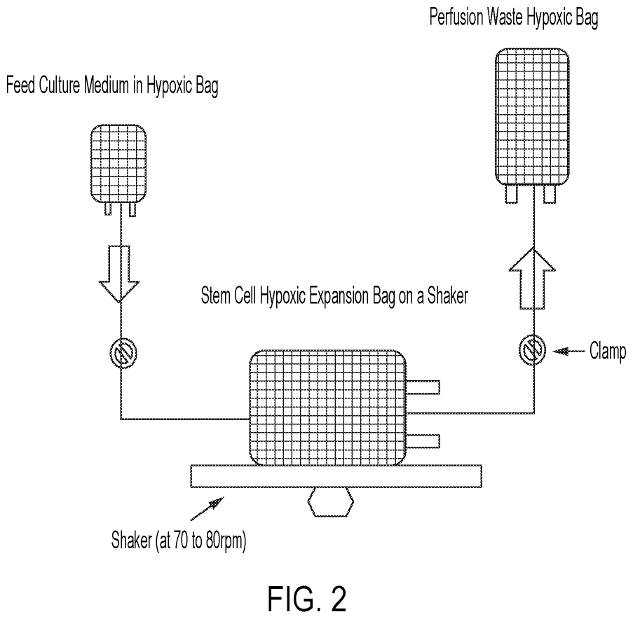

In an aspect of the present disclosure, stem cells are expanded by placing isolated stem cells in a hypoxic stem cell expansion container. In another aspect, the hypoxic stem cell expansion container is placed on a shaker (linear or orbital) and connected to a hypoxic bag comprising a feed culture medium and a hypoxic bag for perfusion waste as provided in FIG. 2. In another aspect, the shaker is set to between 70 and 80 rpm for a period of time to increase mixing and oxygen and carbon dioxide depletion. In another aspect, the hypoxic stem cell expansion container is attached to oxygen and carbon dioxide adsorbents in a closed system. In other aspects, the oxygen and carbon dioxide are controlled and maintained during expansion in a controlled atmosphere by replacing the oxygen with an inert gas such as nitrogen.

In an aspect of the present disclosure, stem cells are expanded by placing isolated stem cells (i.e., stem cells eluted from a stem cell recovery filter) in a hypoxic stem cell expansion container with stem cell expansion medium, placing the hypoxic stem cell expansion container on shaker (linear or orbital) and mixing the stem cells to increase oxygen and carbon dioxide depletion. Typically, the mixing occurs at speed of between 70 and 80 rpm for a period of time but the selection of a suitable mixing methods and speeds are known to a person of skill in the art. In an aspect, the hypoxic stem cell expansion container is maintained at a partial pressure of oxygen that is less than 3,000 pascal (Pa) under standard temperature and pressure (STP) during said expansion culture period of 1 to 5 days. In another aspect, the hypoxic stem cell expansion container is maintained at a partial pressure of oxygen that is less than 2,500 pascal (Pa) under standard temperature and pressure (STP) during said expansion culture period of 1 to 5 days. In another aspect, the hypoxic stem cell expansion container is maintained at a partial pressure of oxygen that is less than 2,000 pascal (Pa) under standard temperature and pressure (STP) during said expansion culture period of 1 to 5 days. In another aspect, the hypoxic stem cell expansion container is maintained at a partial pressure of oxygen that is less than 1,600 pascal (Pa) under standard temperature and pressure (STP) during said expansion culture period of 1 to 5 days. In another aspect, the hypoxic stem cell expansion container is maintained at a partial pressure of oxygen that is less than 1,400 pascal (Pa) under standard temperature and pressure (STP) during said expansion culture period of 1 to 5 days. In another aspect, the hypoxic stem cell expansion container is maintained at a partial pressure of oxygen that is less than 1,200 pascal (Pa) under standard temperature and pressure (STP) during said expansion culture period of 1 to 5 days.

In another aspect, the hypoxic stem cell expansion container is maintained at a partial pressure of oxygen that is less than 3,000 Pa for a culture period of between 1 to 5 days and cultured to expand the cells by between 3- and 14-fold. In another aspect, the hypoxic stem cell expansion container is maintained at a partial pressure of oxygen that is less than 2,000 Pa for a culture period of between 1 to 5 days and cultured to expand the cells by between 3-and 14-fold. In another aspect, the hypoxic stem cell expansion container is maintained at a partial pressure of oxygen that is less than 1,600 Pa for a culture period of between 1 to 5 days and cultured to expand the cells by between 3- and 14-fold. In yet another aspect, the hypoxic stem cell expansion container is maintained at a partial pressure of oxygen that is less than 1,400 Pa for a culture period of between 1 to 5 days and cultured to expand the cells by between 3- and 14-fold. In a further aspect, the hypoxic stem cell expansion container is maintained at a partial pressure of oxygen that is less than 1,200 Pa for a culture period of between 1 to 5 days and cultured to expand the cells by between 3- and 14-fold.

In a further aspect, stem cells are cultured under hypoxic expansion conditions for a period of time to expand stem cells by 3- to 14-fold. In an aspect, stem cells are cultured under hypoxic expansion conditions for a period of time to expand stem cells 3- to 16-fold and prepare a population comprising at least 10⁴ cells. In another aspect, an expanded stem cell population is cultured to increase the number of CD34+ cells after 1 to 5 days by at least 2-fold. In an aspect, stem cells are cultured under hypoxic expansion conditions for a period of time to expand the number of CD34+ cells at least 3 fold after 1 to 5 days. In another aspect, stem cells are cultured under hypoxic expansion conditions for a period of time to expand stem cells to a population comprising between 5 and 10% CD34+ cells. In another aspect, stem cells are cultured under hypoxic expansion conditions for a period of time to expand stem cells to a population comprising less than 2, 1, 0.5, 0.05% then 0.005% CD44+ cells. In another aspect, stem cells are expanded by 3- to 14-fold and contain more than 10% CD34+ and less than 2% CD44+ cells. In another aspect, stem cells are cultured under hypoxic expansion conditions for a period of time sufficient to expand stem cells to a population comprising less than 1% CD90+ cells. In an aspect, the expanded stem cells comprise a population of at least 5% CD34+ cells, less than 2% CD44+ cells, and less than 1% CD90+ cells. In an aspect, stem cells cultured under hypoxic expansion conditions for a period of 1 to 5 days maintain a viability of greater than 98% as measured by cell staining techniques (i.e., trypan blue and 7-amino- actinomycin D (7-AAD).

The present disclosure provides for a hypoxic stem cell expansion container comprising a total surface area of between 0.155 to 0.7 square meters (m²). In another aspect, the hypoxic stem cell expansion container comprises a total surface area of between 0.155 to 0.31 m². In another aspect, the hypoxic stem cell expansion container comprises a total surface area of at least 0.155 m². In another aspect, the hypoxic stem cell expansion container comprises a total surface area of at least 0.2 m². In another aspect, the hypoxic stem cell expansion container comprises a total surface area of at least 0.3 m². In yet another aspect, the hypoxic stem cell expansion container comprises a total surface area of at least 0.4 m². In another aspect, the hypoxic stem cell expansion container comprises a total surface area of at least 0.5 m². In a further aspect, the hypoxic stem cell expansion container comprises a total surface area of at least 0.6 m². In a further aspect, the hypoxic stem cell expansion container comprises a total surface area of less than 0.7 m².

In another aspect, the present disclosure provides for hypoxic stem cell expansion containers that comprise microcarrier beads. In another aspect, the present disclosure provides for hypoxic stem cell expansion containers that comprise microcarrier beads and hypoxic expansion medium.

In general, media suitable for stem cell expansion using the hypoxic methods of the present disclosure are known in the art. Typically, a cell culture medias are buffered medias having a pH between 6.6 and 7.8, include an energy source, (typically glucose/dextrose), and serum proteins (e.g., albumin). In aspects of the present disclosure, the stem cell expansion media are supplemented with additives or growth factors to enhance stem cell expansion. Examples of common stem cell expansion media are StemSpan™ (Stem Cell Technologies, Cambridge, MA), and Stemline (Sigma-Aldrich). In an aspect, the stem cell expansion medium comprises one or more growth factors selected from the group consisting of basic fibroblast growth factor (bFGF), activin A, Activin B, TGF-beta, VEGF, granulocyte colony stimulating factor (G-CSF), granulocyte-macrophage-CSF (GM-CSF), and stem cell factor (SCF). In another aspect, the stem cell expansion medium comprises two or more, three or more, four or more, five or more, or six or more growth factors selected from the group consisting of basic fibroblast growth factor (bFGF), activin A, activin B, TGF-beta, VEGF, granulocyte colony stimulating factor (G-CSF), granulocyte-macrophage-CSF (GM-CSF), and stem cell factor (SCF). In another aspect, the stem cell expansion medium comprises basic fibroblast growth factor (bFGF). In another aspect, the stem cell expansion medium comprises activin A. In another aspect, the stem cell expansion medium comprises Activin B. In another aspect, the stem cell expansion medium comprises TGF-beta. In another aspect, the stem cell expansion medium comprises VEGF. In another aspect, the stem cell expansion medium comprises granulocyte colony stimulating factor (G-CSF). In another aspect, the stem cell expansion medium comprises granulocyte—macrophage-CSF (GM-CSF). In another aspect, the stem cell expansion medium comprises stem cell factor (SCF). In yet another aspect, the stem cell expansion medium comprises one or more, two or more, three or more, four or more, or five or more growth factors and a pH of between 6.6 and 7.8. In another aspect, the stem cell expansion medium comprises a pH of at least 6.6. In yet another aspect, the stem cell expansion medium comprises a pH of at least 7. In a further aspect, the stem cell expansion medium comprises a pH of 7.5.

In another aspect, the present disclosure provides for a stem cell expansion medium further comprising one or more additives selected from the group consisting of retinoic acid, ascorbic acid, hormones, intracellular cAMP elevating agents, Flt-3 ligand, combinations of different cytokines and growth factors, such as SCF, Flt3, TPO, IL-3, and IL-6. In another aspect, the stem cell expansion medium further comprising two or more, three or more, four or more, or five or more additives selected from the group consisting of retinoic acid, ascorbic acid, hormones, intracellular cAMP elevating agents, Flt-3 ligand, combinations of different cytokines and growth factors. In yet another aspect, the stem cell expansion medium comprises retinoic acid. In yet another aspect, the stem cell expansion medium comprises ascorbic acid. In another aspect, the stem cell expansion medium comprises hormones. In yet another aspect, the stem cell expansion medium comprises intracellular cAMP elevating agents. In another aspect, the stem cell expansion medium comprises Flt-3 ligand. In yet another aspect, the stem cell expansion medium comprises one or more growth factors selected from the group consisting of SCF, Flt3, TPO, IL-3, and IL-6. In an aspect of the present disclosure, a stem cell expansion medium comprises one or more growth factors, one or more additives, and a pH of between 6.6 and 7.8.

The present disclosure provides for, and includes, collecting a blood product containing stem cells into an oxygen absorbing environment. In an aspect, an oxygen absorbing environment is a hypoxic collection container comprising an oxygen barrier layer. In another aspect, an oxygen absorbing environment is a hypoxic collection container comprising an oxygen barrier layer enclosing an oxygen permeable layer. In another aspect, an oxygen absorbing environment is a hypoxic collection container comprising an oxygen permeable layer in contact with the blood product layer on one side and an oxygen sorbent on the other side, and an oxygen barrier layer enclosing both the oxygen sorbent and the oxygen permeable layer. In yet another aspect, an oxygen absorbing environment is a hypoxic collection container comprising an oxygen permeable layer, one or more oxygen or oxygen and carbon dioxide sorbents, and an oxygen barrier layer. The present disclosure also provides for, and includes, a hypoxic storage container for

storing stem cells or red blood cells. In an aspect, a hypoxic storage container comprises an oxygen barrier layer. In another aspect, a hypoxic storage container comprises an oxygen barrier layer enclosing an oxygen permeable layer. In another aspect, a hypoxic storage container comprises an oxygen permeable layer in contact with the blood product layer on one side and an oxygen sorbent on the other side, and an oxygen barrier layer enclosing both the oxygen sorbent and the oxygen permeable layer. In yet another aspect, a hypoxic storage container comprises an oxygen permeable layer, one or more oxygen or oxygen and carbon dioxide sorbents, and an oxygen barrier layer.

The present disclosure also provides for, and includes, an oxygen barrier layer that is substantially impermeable to oxygen or oxygen and carbon dioxide. In an aspect, an oxygen barrier bag is prepared from flexible film materials. In another aspect, the oxygen barrier layer is prepared from a stiff, or inflexible film material. As provided herein, the term oxygen barrier refers to materials and compositions that provide a barrier to the passage of oxygen from one side of the barrier to the other, sufficient to prevent significant increases in the partial pressure of carbon dioxide over 42 days or more.

Unless indicated otherwise, an oxygen barrier refers to membranes that are substantially impermeable to oxygen. As used herein, substantially impermeable to oxygen is a permeability to oxygen of less than about 1.0 cc of oxygen per square meter per day. In another aspect, substantially impermeable to oxygen is a permeability to oxygen of less than about 0.5 cc of oxygen per square meter per day. In certain aspect, a membrane suitable for use in the preparation of an oxygen barrier layer is a material characterized by a Barrer value of less than about 0.140 Barrer. As used herein, an oxygen barrier layer is also substantially impermeable to carbon dioxide. An oxygen barrier layer substantially impermeable to carbon dioxide means a the layer allows no more than 10 cc of carbon dioxide inside the receptacle over a period of 3 months, and more preferably no more than 5 cc of carbon dioxide over a period of 6 months.

Materials and methods to prepare a gas impermeable barrier bag are known in the art. See, for example, U.S. Pat. 7,041,800 issued to Gawryl et al., U.S. Pat. 6,007,529 issued to Gustafsson et al., and U.S. Patent Application Publication No. 3013/0327677 by McDorman, each of which are hereby incorporated by reference in their entireties. Materials and methods for preparing an oxygen absorbing environment and hypoxic collection and storage containers are also known in the art. See, U.S Pat. No. 9,801,784 issued to Yoshida et al., U.S Pat. No. 10,849,824 to Yoshida et al., and U.S. Pat. No. 10,058,091 to Wolf et al., each of which are hereby incorporated by reference in their entireties. Impermeable materials are routinely used in the art and any suitable material can be used. In the case of molded polymers, additives are routinely added to enhance the oxygen and carbon dioxide barrier properties. See, for example, U.S. Pat. No. 4,837,047 issued to Sato et al. For example, U.S. Pat. No. 7,431,995 issued to Smith et al. describes an oxygen- and carbon dioxide-impermeable receptacle composed of layers of ethylene vinyl alcohol copolymer and modified ethylene vinyl acetate copolymer, impermeable to oxygen and carbon dioxide ingress. In another aspect, the gas impermeable barrier bag is impermeable to oxygen and carbon dioxide.

In certain aspects, films that are substantially impermeable to carbon dioxide, oxygen, or both carbon dioxide and oxygen are laminated films. In an aspect, a laminated film that is substantially impermeable to carbon dioxide, oxygen, or both carbon dioxide and oxygen is a laminated foil film. Film materials can be polymers or multilayer constructions that are combinations of foils and polymers. In an aspect, a laminated film is a polyester membrane laminated with aluminum. An example of suitable aluminum laminated film, also known as a laminated foil, that is substantially impermeable to oxygen is known in the art. For example, U.S. Pat. No. 4,798,728 to Sugisawa discloses aluminum laminated foils of nylon, polyethylene, polyester, polypropylene, and vinylidene chloride. Other laminated films are known in the art. For example, U.S. Pat. No. 7,713,614 to Chow et al. discloses multilayer containers comprising an ethylene-vinyl alcohol copolymer (EVOH) resin that is substantially impermeable to oxygen. In an aspect, a gas impermeable barrier bag is a barrier bag constructed by sealing three or four sides by means of heat sealing. The bag is constructed of a multilayer construction that includes materials that provide enhancement to carbon dioxide and oxygen barrier properties. The bag is constructed of a multilayer construction that includes materials that provide enhancement to carbon dioxide and oxygen barrier properties. Such materials include the Rollprint Clearfoil® V2 film, having an oxygen transmission rate of 0.01 cc/100 in²/24 hrs., Rollprint Clearfoil® X film, having an oxygen transmission rate of 0.004 cc/100 in²/24 hrs., and Clearfoil® Z film having an oxygen transmission rate of 0.0008 cc/100 in²/24 hrs., (Rollprint Packaging Products, Addison, IL). Other manufacturers make similar products with similar oxygen transmission rates, such as Renolit Solmed Wrapflex ° films (American Renolit Corp., City of Commerce, CA). An example of suitable aluminum laminated film, also known as a laminated foil, that is substantially impermeable to oxygen is obtainable from Protective Packaging Corp. (Carrollton, TX).

In an aspect of the present disclosure, the hypoxic collection container comprises a partial pressure of oxygen (pO₂) of less than 1350 Pa. In another aspect, the hypoxic collection container comprises a partial pressure of carbon dioxide (pCO₂) of less than 400 Pa. In another aspect, the hypoxic collection container comprises a partial pressure pO₂ of between 990 and 3000 Pa, between 990 and 2500 Pa, between 990 and 2000 Pa, between 990 and 1500 Pa, or between 1000 and 3000 Pa. In another aspect of the present disclosure, the hypoxic collection container comprises a pO₂ of between 400 and 2000 Pa, between 500 and 2000 Pa, between 600 and 2000 Pa, between 700 and 2000 Pa, between 800 and 2000 Pa, between 900 and 2000 Pa, between 900 and 2000 Pa, between 1000 and 2000 Pa, between 1200 and 2000 Pa, between 1400 and 2000 Pa, between 1600 and 2000 Pa, between 1800 and 2000 Pa.

In another aspect, the hypoxic storage container comprises a partial pressure of oxygen (pO₂) of less than 1350 Pa. In another aspect, the hypoxic storage container comprises a partial pressure of carbon dioxide (pCO₂) of less than 400 Pa. In another aspect, the hypoxic storage container comprises a partial pressure pO₂ of between 990 and 3000 Pa, between 990 and 2500 Pa, between 990 and 2000 Pa, between 990 and 1500 Pa, or between 1000 and 3000 Pa. In another aspect of the present disclosure, the hypoxic storage container comprises a pO₂ of between 400 and 2000 Pa, between 500 and 2000 Pa, between 600 and 2000 Pa, between 700 and 2000 Pa, between 800 and 2000 Pa, between 900 and 2000 Pa, between 900 and 2000 Pa, between 1000 and 2000 Pa, between 1200 and 2000 Pa, between 1400 and 2000 Pa, between 1600 and 2000 Pa, between 1800 and 2000 Pa.

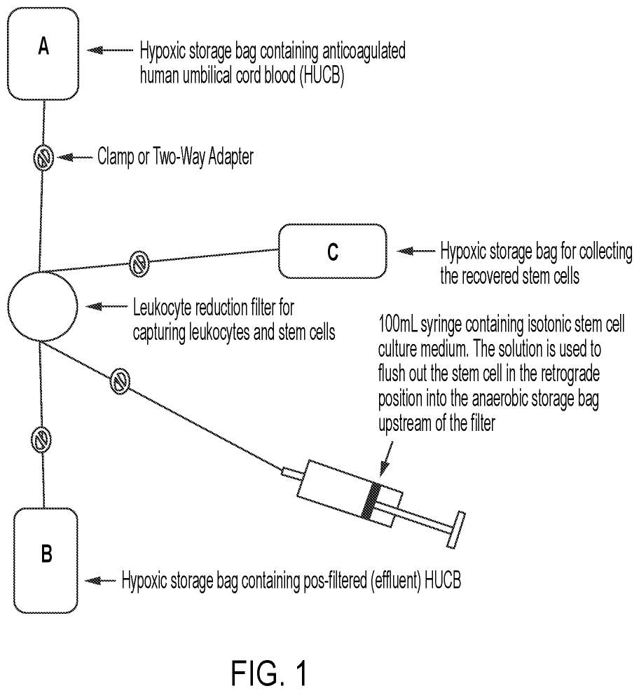

The present disclosure provides for, and includes, methods for hypoxic collection of a blood product and isolation of stem cells. In aspects, the blood product is collected in a hypoxic collection container and filtered to isolate and concentrate stem cells. The stem cells are isolated using a stem cell or stem cell and leukocyte filter. Stem cells are eluted from the filter into a hypoxic storage bag or hypoxic stem cell expansion container using an isotonic media as shown in FIG. 1. In an aspect, the isotonic medium comprises 10 to 30 mM phosphate buffered saline with 0.5mM calcium chloride, 1 mM magnesium sulfate containing either 250-500 mM trehalose, 3% dextran 40, or 3% dextran 70 and has a tonicity of 280-300 mOsmol/kg of water. In an aspect, the isotonic media is a commercially available medium, Plasma-Lyte (Baxter, Deerfield, IL), HypoThermosol (BioLife Solutions Inc., Bothell, WA). In another aspect, the isotonic media further comprises one or more antioxidants selected from the group consisting of 1 mM N-acetyl cysteine, 1 mM trolox-water soluble vitamin E, 1 mM vitamin C, or combinations thereof. In another aspect, the isotonic stem cell culture media further comprises two or more antioxidants selected from the group consisting of 1 mM N-acetyl cysteine, 1 mM trolox-water soluble vitamin E, 1 mM vitamin C, or combinations thereof.

In another aspect, the present disclosure provides for, equilibrating the isotonic media with oxygen prior to stem cell elution.

The present disclosure provides for, and includes, kits for processing stem cells for storage or transplantation comprising an hypoxic collection container, a leukocyte and stem cell recovery filter, a first accessory hypoxic storage container for collecting filtered stem cells depleted blood product, and a second accessory hypoxic storage container comprising stem cell maintaining medium. In an aspect of the present disclosure, the second accessory hypoxic storage container is in fluid communication with said leukocyte and stem cell recovery filter. In another aspect, the kit comprises less than 1,400 Pa partial pressure of oxygen (pO₂). In another aspect, the leukocyte and stem cell filter is a single combined filter. In another aspect, the first accessory hypoxic storage container comprises an outer receptacle substantially impermeable to oxygen, an inner collapsible container permeable to gas, an oxygen or oxygen and carbon dioxide sorbent situated between the outer receptacle and the inner collapsible container, and at least one inlet/outlet that is substantially impermeable and passing through the outer receptacle and that is in fluid communication with the collapsible container. In another aspect, the kit comprises less than 1,400 Pa pO₂. In another aspect, the second accessory hypoxic storage container comprises an outer receptacle substantially impermeable to oxygen, an inner collapsible container permeable to gas, an oxygen or oxygen and carbon dioxide sorbent situated between the outer receptacle and the inner collapsible container, and at least one inlet/outlet that is substantially impermeable and passing through the outer receptacle and that is in fluid communication with the collapsible container. In another aspect, hypoxic collection and storage containers include those known and described in International Publication Nos. WO 2016/172645 and WO 2016/145210 to Hemanext Inc, both of which are incorporated herein by reference.

The present disclosure provides for, and includes, methods of preparing stem cells for transplantation comprising exposing an hypoxic storage bag comprising frozen stem cells to a temperature of at least 25° C. to form thawed stem cells; centrifuging the thawed stem cells and removing the supernatant; and resuspending the stem cells in a solution comprising 3% dextran 40. In another aspect, the present disclosure provides for, and includes, methods for preparing stem cells for transplantation in a subject in need thereof comprising: thawing hypoxic prepared stem cells; expanding the stem cells in a hypoxic stem cell expansion system as described above; and transplanting the expanded stem cells into the subject in need thereof. In another aspect, at least 10⁴ total HSCs are transplanted in a subject in need thereof. In yet another aspect, at least 10⁵, 10⁶, 10⁷, 10⁸, or 10⁹ total HSCs are transplanted into a subject in need thereof.

In another aspect, the stem cell expansion system comprises an outer receptacle substantially impermeable to oxygen, an inner collapsible container, an oxygen or oxygen and carbon dioxide sorbent situated between the outer receptacle and the inner collapsible blood container, and at least one inlet/outlet that is substantially impermeable and passing through the outer receptacle and that is in fluid communication with the collapsible container. In another aspect, the stem cell expansion system comprises 3D cultivation of stem cells in a controlled environment comprising a pO₂ of less than 1400 Pa. In yet another aspect, the system comprises a 2D cultivation of stem cells in a controlled environment comprising a pO₂ of less than 1400 Pa

The present disclosure provides for, and includes, methods for preparing stem cells for transplantation comprising collecting human umbilical cord blood (HUCB), separating leukocytes and stem cells with a leukocyte reduction filter and a stem cell retaining filter, eluting the stem cells from the stem cell retaining filter with an isotonic media, and transferring the stem cells into a hypoxic storage container.

In another aspect, the present disclosure provides methods for preparing stem cells for transplantation comprising exposing a hypoxic storage container comprising frozen hypoxic stem cells to 37° C.; immersing the hypoxic storage bag comprising frozen hypoxic stem cells in a water bath less than 42° C. for thawing said frozen stem cells to generate thawed stem cells; diluting the thawed stem cells with equal volume of hypoxic solution containing 2.5% (wt/vol) human albumin to form diluted stem cells. In another aspect, the present disclosure provides for diluting the thawed stem cells with equal volume of hypoxic solution containing from 1 to 5% (wt/vol) human albumin to form diluted stem cells. In another aspect, the hypoxic solution contains at least 1, 1.5, 2, 2.5, 3, 3.5, 4, 4.5, and 5% (wt/vol) human albumin. In another aspect, the hypoxic solution contains between 1 and 2%, between 1 and 3%, between 1 and 4%, between 1 and 5%, between 2 and 3%, between 2 and 5%, between 2 and 4%. In yet another aspect, the hypoxic solution contains less than 5%, less than 4.5%, less than 4%, less than 3.5%, less than 3.0%, less than 2.5%, less than 2.0%, and less than 1.5%. In yet another aspect, the hypoxic solution contains less than 5%, less than 4.5%, less than 4%, less than 3.5%, less than 3.0%, less than 2.5%, less than 2.0%, and less than 1.5%.

In another aspect, the hypoxic solution further comprises 5% Dextran 40. In another aspect, stem cells are exposed to 37° C. for 15 to 30 mins. In another aspect, the hypoxic storage bag comprising frozen stem cells are placed in a water bath of at least 32° C. In yet another aspect, methods for preparing stem cells further comprise centrifuging the diluted stem cells and removing supernatant. In another aspect, the present disclosure provides methods for preparing stem cells for transplantation that further comprise resuspending said thawed stem cells in an albumin dextran solution to a volume appropriate for transplantation.

In an aspect of the present disclosure, the hypoxic collection container, the leukocyte filter, the stem cell recovery filter, and the hypoxic storage container are part of a system comprising a pO₂ of less than 1400 Pa (10 mmHg). More specifically, the filters, devices, solutions, and other components are part of a system that is maintained in a hypoxic state. In another aspect, the hypoxic collection container, the leukocyte filter, the stem cell recover filter, and the hypoxic storage container are part of a system comprising a pO₂ of less than 1350 Pa (10 mmHg). In another aspect, the hypoxic collection container, the leukocyte filter, the stem cell recovery filter, and the hypoxic storage container are in a system comprising a pO₂ of between 990 and 3000 Pa, between 990 and 2500 Pa, between 990 and 2000 Pa, between 990 and 1500 Pa, or between 1000 and 3000 Pa. In another aspect of the present disclosure, the hypoxic collection container, the leukocyte filter, the stem cell recovery filter, and the hypoxic storage container are in a system comprising a pO₂ of between 400 and 2000 Pa, between 500 and 2000 Pa, between 600 and 2000 Pa, between 700 and 2000 Pa, between 800 and 2000 Pa, between 900 and 2000 Pa, between 900 and 2000 Pa, between 1000 and 2000 Pa, between 1200 and 2000 Pa, between 1400 and 2000 Pa, between 1600 and 2000 Pa, between 1800 and 2000 Pa.

In an aspect, the present disclosure provides for a hypoxic collection bag that is connected to two accessory hypoxic storage bags in a closed hypoxic system. In an aspect, the present disclosure provides for interconnected bags being centrifuged in a closed system. In another aspect, the interconnected bags are centrifuged and the supernatant is expressed into one of the attached bags.

The present disclosure provides for, methods for preparing stem cells for transplantation comprising collecting a blood product comprising stem cells; separating leukocytes and stem cells from the blood product; transferring the stem cells into a hypoxic storage container comprising an O₂ barrier characterized by an oxygen (O₂) permeability of less than 0.5 cc of oxygen per square meter per day, and storing the stem cells in a hypoxic environment of less than 3000 Pa O₂ for a period of time at a temperature. In an aspect of the present disclosure, the storage period is at least 2, 4, 7, 10, 14, 21, or 28 days. In another aspect, the leukocytes and stem cells are separating from the blood product using a leukocyte filter, stem cell recovery filter, or a combined leukocyte and stem cell recover filter as described in the present disclosure. In another aspect, the stem cells are eluted from the stem cell recovery filter or leukocyte and stem cell recover filter with an isotonic media prior to being stored in a hypoxic environment. In another aspect, the hypoxic environment is a hypoxic storage container as provided in the present disclosure. In another aspect, the blood product is collected in a hypoxic collection container comprising an O₂ barrier characterized by an oxygen (O₂) permeability of less than 0.5 cc of oxygen per square meter per day and an oxygen sorbent. In another aspect, the hypoxic collection container comprising an O₂ barrier characterized by an oxygen (O₂) permeability of less than 25 Barrer. The blood product in the hypoxic collection container is mixed until the initial partial pressure of oxygen (pO₂) of the blood product is reduced by between 20 and 60%. In another aspect, the pO₂ is reduced by between 80 and 95%. In another aspect, the pO₂ is reduced by between 50 and 95%. In another aspect, the pO₂ is reduced by between 70 and 95%. In another aspect, the pO₂ is reduced by between 50 and 95%. In a further aspect, the stem cells are separated from the blood product prior to mixing.

The present disclosure also provides for methods which do not expose stem cells to normoxic conditions for greater than one hour between steps of collecting and transferring to hypoxic storage container or a hypoxic stem cell expansion container. In an aspect, normoxic conditions comprise a partial pressure of oxygen at least about 157 mmHg (or 21,000 Pa).

All references provided throughout the present disclosure are hereby incorporated by reference in their entireties.

The present disclosure also provides for the following embodiments:

Embodiment 1. A method for preparing stem cells for transplantation into a patient in need thereof comprising: collecting a blood product containing stem cells into an oxygen absorbing environment comprising an hypoxic collection container comprising an O₂ barrier characterized by an oxygen (O₂) permeability of less than 0.5 cc of oxygen per square meter per day and an oxygen sorbent; mixing the blood product until the initial partial pressure of oxygen (pO₂) of the blood product is reduced by between 80 and 95% separating leukocytes and stem cells from said blood product comprising applying said blood product to a filter wherein the stem cells and leukocytes are retained on said filter to prepare a leukocyte and stem cells depleted blood product; eluting said stem cells from said filter with an isotonic media; and
transferring said stem cells into a hypoxic storage container comprising an O₂ barrier characterized by an oxygen (O₂) permeability of less than 0.5 cc of oxygen per square meter per day and storing said cells to prepare hypoxic stored stem cells.

Embodiment 2. The method of embodiment 1, further comprising transferring said stem cells to an hypoxic stem cell expansion container before or after said transferring to said hypoxic storage container and expanding said stem cells to form an expanded stem cell population.

Embodiment 3. The method of embodiment 2, further comprising transplanting said expanded stem cells into a patient in need thereof

Embodiment 4. The method of embodiment 1, wherein said mixing, separating, eluting, and transferring are each performed under a pO₂ of between 400 and 2000 Pa.

Embodiment 5. The method of embodiment 1, wherein said mixing is for up to 3 hours.

Embodiment 6. The method of embodiment 1, wherein said mixing is until the initial partial pressure of oxygen (pO₂) of said blood product is reduced by at least 50%.

Embodiment 7. The method of embodiment 1, wherein said isotonic media is deoxygenated isotonic media comprising a pO₂ of at least 5333 Pa.

Embodiment 8. The method of embodiment 1, wherein said transferring comprises directly eluting into said hypoxic storage container.

Embodiment 9. The method of embodiment 1, wherein said eluting comprises 50 to 200 mL of said isotonic media.

Embodiment 10. The method of embodiment 1, further comprising equilibrating said isotonic media with oxygen in the air.

Embodiment 11. The method of embodiment 1, wherein said hypoxic storage container comprises an outer receptacle substantially impermeable to oxygen, a collapsible inner blood container, an oxygen or oxygen and carbon dioxide sorbent situated between said outer receptacle and said inner collapsible blood container, and at least one inlet/outlet that is substantially impermeable and passing through said outer receptacle and that is in hypoxic fluid communication with said collapsible container, wherein said hypoxic storage container comprises less than 1,400 Pa pO₂.

Embodiment 12. The method of embodiment 1, wherein said blood product is selected from the group consisting of peripheral blood, human umbilical cord blood (HUCB), and bone marrow.

Embodiment 13. The method of embodiment 1, wherein said stem cells are induced pluripotent stem cells (iPSC) from blood cells.

Embodiment 14. The method of embodiment 1, wherein said hypoxic collection container contains an anticoagulant.

Embodiment 15. The method of embodiment 1, wherein said filter comprises a leukocyte reduction filter and a stem cell retaining filter.

Embodiment 16. The method of embodiment 1, wherein red blood cells contained in said blood product comprises a red blood cell oxygen saturation of less than 20%, 15%, 10%, 5%, 1% or 0.01% saturated O₂(sO₂) during said separating on said filter.

Embodiment 17. The method of embodiment 1, wherein said hypoxic collection container, said filter, and said hypoxic storage container are in a system comprising a pO₂ of less than 1350 Pa.

Embodiment 18. The method of embodiment 1, wherein said filter is maintained under hypoxic conditions comprising a partial pressure of oxygen (pO₂) of less than 6000 Pa.

Embodiment 19. The method of embodiment 1, wherein said hypoxic collection container or said hypoxic storage container comprises a partial pressure of oxygen (pO₂) of less than 1350 Pa.

Embodiment 20. The method of embodiment 1, wherein said hypoxic collection container, said hypoxic storage container, or both said hypoxic container and said hypoxic storage container comprise a partial pressure of carbon dioxide (pCO₂) of less than 6000 Pa.

Embodiment 21. The method of embodiment 1, wherein said leukocyte reduction filter and said stem cell retaining filter are a combined leukocyte and stem cell filter.

Embodiment 22. The method of embodiment 14, wherein said anticoagulant is citrate-phosphate dextrose (CPD), citrate-phosphate-dextrose-adenine 1 (CPDA-1), acid citrate dextrose (ACD) or heparin.

Embodiment 23. The method of embodiment 1, wherein said hypoxic collection container is in fluid communication with said filter and said filter is in fluid communication with said hypoxic storage container, wherein the combination comprises less than 1,400 Pa pO₂.

Embodiment 24. The method of embodiment 15, wherein said filter comprises a stem cell specific monoclonal antibody.

Embodiment 25. The method of embodiment 1, wherein said hypoxic stored stem cells are maintained at a partial pressure of oxygen that is less than 1400 Pascal.

Embodiment 26. The method of embodiment 1, further comprising determining the total number of nucleated cells in said blood product, in said eluted stem cells, in said hypoxic stored stem cells, or combinations thereof

Embodiment 27. The method of embodiment 26, wherein said determining of the numbers of cells is after said collecting.

Embodiment 28. The method of embodiment 26 or 27, wherein said determining of the numbers of cells is after said filtering.

Embodiment 29. The method of embodiment 28, wherein the total number of nucleated cells in said eluted stem cells comprises at least 50% of the total number of nucleated cells in said blood product.

Embodiment 30. The method of embodiment 1, wherein said stem cells are hematopoietic stem cells (HSCs).

Embodiment 31. The method of embodiment 30, wherein said hematopoietic stem cells comprise one or more hematopoietic stem cell markers selected from the group consisting of polycomb group RING finger protein 4 (BMI-1), cluster of differentiation 21 (CD21), cluster of differentiation 22 (CD22), cluster of differentiation 34 (CD34), cluster of differentiation 38 (CD38), cluster of differentiation 41 (CD41), cluster of differentiation 44 (CD44), cluster of differentiation 45 (CD45), cluster of differentiation 48 (CD48), cluster of differentiation 90 (CD90; Thy 1), cluster of differentiation 105 (CD105), cluster of differentiation 106 (CD106), cluster of differentiation 117 (CD117; c-kit), cluster of differentiation 127 (CD127), cluster of differentiation 150 (CD150), c-myc, endothelial protein c receptor (EPCR), lymphocyte antigen-6 (Ly6A/E; sca-1), MYB, induced myeloid leukemia cell differentiation protein (Mcl-1), phosphatase and tensin homolog (PTEN), Skp, Cullin, F-box (SCF; kit ligand), single transducer and activator of transcription 5a (STAT5a), single transducer and activator of transcription 5b (STAT5b), and vascular endothelial growth factor receptor 2 (VEGFR2).

Embodiment 32. The method of embodiment 30, further comprising transplanting at least 10 4 hypoxic stored stem cells in said patient in need thereof

Embodiment 33. The method of embodiment 2, wherein said hypoxic stem cell expansion container comprises a total surface area of between 0.155 to 0.7 square meters (m²).

Embodiment 34. The method of embodiment 33, wherein said hypoxic stem cell expansion container comprises a total surface area of 0.155 to 0.31 m².

Embodiment 35. The method of embodiment 33, wherein said hypoxic stem cell expansion container comprises microcarrier beads and stem cell expansion medium.

Embodiment 36. The method of embodiment 35, wherein said stem cell expansion medium comprises growth factors selected from the group consisting of basic fibroblast growth factor (bFGF), activin A, Activin B and TGF-beta, VEGF, granulocyte colony stimulating factor (G-CSF), granulocyte—macrophage- CSF (GM-CSF), and stem cell factor (SCF).

Embodiment 37. The method of embodiment 35, wherein said stem cell expansion medium comprises a pH of between 6.6 and 7.8.

Embodiment 38. The method of embodiment 35, wherein said stem cell expansion medium comprises additives selected from the group consisting of retinoic acid, ascorbic acid, hormones, or intracellular cAMP elevating agents, Flt-3ligand, combinations of different cytokines and growth factors, such as SCF, Flt3, TPO, IL-3, and IL-6.

Embodiment 39. The method of embodiment 33, wherein said hypoxic stem cell expansion container is maintained at a partial pressure of oxygen that is less than 3,000 pascal (Pa) under standard temperature and pressure (STP) during said expansion.

Embodiment 40. The method of embodiment 39, wherein said pO₂ during said hypoxic stem cell expansion is maintained at a partial pressure of oxygen that is less than 1,400 Pascal under STP.

Embodiment 41. The method of embodiment 33, wherein said expanding stem cells comprises culturing under hypoxic conditions comprising a pO₂ of less than 1,400 pascal under STP, for a culture period of 3 to 4 days to prepare an expanded hypoxic stem cell population.

Embodiment 42. The method of embodiment 41, wherein said expanding said hypoxic stem cells comprises culturing for a period to increase said stem cells from 3- to 14-fold.

Embodiment 43. The method of embodiment 41, wherein said expanding comprises culturing for a period sufficient to expanding said hypoxic stem cells to prepare a population comprising at least 10 4 cells.

Embodiment 44. The method of embodiment 41, wherein said expanded stem cell population comprises at least a 2-fold increase in CD34+ cells after said expanding.

Embodiment 45. The method of embodiment 44, wherein said expanded stem cell population comprises at least a 3-fold increase in CD34+ cells after said expanding.

Embodiment 46. The method of embodiment 45, wherein said expanded stem cell population comprises between 5 and 10% CD34+ cells.

Embodiment 47. The method of embodiment 41, wherein said expanded stem cell population comprises less than 2, 1, or 0.005% CD44+ cells.

Embodiment 48. The method of embodiment 41, wherein said expanded stem cell population less than 1% CD90+ cells.

Embodiment 49. The method of embodiment 33, wherein said stored stem cells are mesenchymal stem cells (MSCs) comprising one or more markers selected from the group consisting CD44, CD90, CD105, CD106, CD166, and Stro-1.

Embodiment 50. The method of embodiment 41, wherein said expanded stem cell population comprises a viability of greater than 98%.

Embodiment 51. The method of embodiment 50, wherein said viability is measured by trypan blue exclusion assay.

Embodiment 52. The method of embodiment 50, wherein said viability is measured by cell exclusion of 7-amino-actinomycin D (7-AAD).

Embodiment 53. The method of embodiment 1, wherein said hypoxic stored stem cells maintain a viability greater than stem cells collected, stored, and cultured under normoxic conditions, wherein said normoxic conditions comprise at least about 21,000 pascal partial pressure of oxygen.

Embodiment 54. A method for preparing stem cells for transfusion comprising:
collecting a blood product comprising stem cells; separating leukocytes and stem cells from said blood product; transferring said stem cells into a hypoxic storage container comprising an O₂ barrier characterized by an oxygen (O₂) permeability of less than 0.5 cc of oxygen per square meter per day, and storing said stem cells in a hypoxic environment at a partial pressure of oxygen of less than 3500 Pa for a period of time at a temperature of less than 37° C.

Embodiment 55. The method of embodiment 54, wherein said hypoxic storage container comprises an outer receptacle substantially impermeable to oxygen, a collapsible inner blood container, an oxygen or oxygen and carbon dioxide sorbent situated between said outer receptacle and said collapsible inner blood container, and at least one inlet/outlet that is substantially impermeable and passing through said outer receptacle and that is in fluid communication with said collapsible container, wherein said combination comprises less than 1,400 Pa pO₂.

Embodiment 56. The method of embodiment 54, wherein collecting said blood product comprises a hypoxic environment having a partial pressure of oxygen that is less than 3000 Pa.

Embodiment 57. The method of embodiment 54, wherein said blood product is selected from the group consisting of peripheral blood, human umbilical cord blood (HUCB), and bone marrow.

Embodiment 58. The method of embodiment 54, wherein said stem cells are induced pluripotent stem cells (iPSCs).

Embodiment 59. The method of embodiment 54, wherein said separating comprises centrifugation to prepare concentrated stem cells.

Embodiment 60. The method of embodiment 59, further comprising reducing leukocytes before or after said centrifugation.

Embodiment 61. The method of embodiment 59, further comprising removing supernatant from said concentrated stem cells and resuspending said concentrated stem cells in a hypoxic stem cell suspension media.

Embodiment 62. The method of embodiment 61, wherein said stem cell suspension media is diluted with a cryoprotectant selected from the group consisting of trehalose, mannitol, sucrose, ethylene glycol, dimethyl sulfoxide, dextran, hydroxyethyl starch, glucose, glycerol, polyvinyl pyrrolidone, propylene glycol, polyethylene glycol, 2-methyl-2,4-pentanediol, formamide, glycerol-3phosphate, proline, sorbitol, diethyl glycol, triethylene glycol, and combinations thereof.

Embodiment 63. The method of embodiment 62, wherein said storing is at −80° C. or in liquid nitrogen at approximately −195.79° C.

64. The method of embodiment 54, wherein said at least one inlet/outlet further comprises a unitary tube that is substantially impermeable to oxygen comprising a first tubing, a bond, and a second tubing, wherein said unitary tube is a barrier traversing tube having at least one oxygen barrier layer, and at least one blood compatible layer.

Embodiment 65. A method for preparing stem cells for transplantation comprising: collecting human umbilical cord blood (HUCB); separating leukocytes and stem cells from said HUCB by applying the HUCB to a leukocyte reduction filter and a stem cell retaining filter; eluting said stem cells from said stem cell retaining filter with an isotonic media; and transferring said stem cells into an hypoxic storage container.

Embodiment 66. The method of embodiment 65, wherein said isotonic media comprises 10 to 30 mM phosphate buffered saline with 0.5 mM calcium chloride, 1 mM magnesium sulfate containing either 250-500 mM trehalose, 3% dextran 40, or 3% dextran 70 and has a tonicity of 280-300 mOsmol/kg of water.

Embodiment 67. The method of embodiment 66, wherein said isotonic media further comprises an antioxidant selected from the group consisting of 1 mM N-acetyl cysteine, 1 mM trolox-water soluble vitamin E, 1 mM vitamin C, or combinations thereof

Embodiment 68. The method of embodiment 65, wherein said leukocyte reduction filter and said stem cell retaining filter are the same filter.

Embodiment 69. The method of embodiment 65, wherein said method is performed at a partial pressure of oxygen of less than 3500 Pa.

Embodiment 70. A kit for processing stem cells for transplantation comprising: an hypoxic collection container; a leukocyte and stem cell recovery filter; a first accessory hypoxic storage container for collecting filtered supernatant; a second accessory hypoxic storage container comprising stem cell maintaining medium; wherein said second accessory hypoxic storage container is in fluid communication with said leukocyte and stem cell recovery filter, wherein said kit comprises less than 1,400 Pascals (Pa) partial pressure of oxygen (pO₂).

Embodiment 71. The kit of embodiment 70, wherein said leukocyte and stem cell filter is a single filter.

Embodiment 72. The kit of embodiment 70, wherein said first accessory hypoxic storage container comprises an outer receptacle substantially impermeable to oxygen, an inner collapsible blood container, an oxygen or oxygen and carbon dioxide sorbent situated between said outer receptacle and said inner collapsible blood container, and at least one inlet/outlet that is substantially impermeable and passing through said outer receptacle and that is in fluid communication with said collapsible container, wherein said fluid communication comprises less than 1,400 Pa pO₂.

Embodiment 73. The kit of embodiment 70, wherein said second accessory hypoxic storage container comprises an outer receptacle substantially impermeable to oxygen, an inner collapsible blood container, an oxygen or oxygen and carbon dioxide sorbent situated between said outer receptacle and said inner collapsible blood container, and at least one inlet/outlet that is substantially impermeable and passing through said outer receptacle and that is in fluid communication with said collapsible container.

Embodiment 74. The method of embodiment 70, wherein said first and second accessory hypoxic storage container further comprises a spacer selected from the group consisting of a mesh, a molded mat, a woven mat, a non-woven mat, an open cell foam, a strand veil, and a strand mat.

Embodiment 75. A method of preparing stem cells for transplantation comprising: exposing a hypoxic storage container comprising frozen hypoxic stored stem cells to 37° C. ; immersing said hypoxic storage bag comprising frozen hypoxic stored stem cells in a water bath less than 42° C. for thawing said frozen stem cells to generate thawed hypoxic stored stem cells; diluting said thawed hypoxic stored stem cells with equal volume of hypoxic solution containing 2.5% (wt/vol) human albumin to form diluted hypoxic stored stem cells.

Embodiment 76. The method of embodiment 75, wherein said solution further comprises 5% Dextran 40.

Embodiment 77. The method of embodiment 75, wherein said water bath is at least 32° C.

Embodiment 78. The method of embodiment 75, wherein said exposing is for 15 to 30 mins.

Embodiment 79. The method of embodiment 75, further comprising centrifuging said diluted stem cells and removing supernatant.

Embodiment 80. The method of embodiment 75, further comprising resuspending said thawed hypoxic stored stem cells in an albumin dextran solution to a volume appropriate for transplantation.

Embodiment 81. The method of embodiment 80, wherein said volume appropriate for transplantation is between 10 and 50 milliliters per kilogram (mL/kg).

Embodiment 82. A method of preparing stem cells for transplantation comprising: exposing an hypoxic storage bag comprising frozen hypoxic stored stem cells to at least 25° C. to form thawed hypoxic stored stem cells;

centrifuging said thawed hypoxic stored stem cells and removing the supernatant; and resuspending said hypoxic stored stem cells in a solution comprising 3% dextran 40, wherein the hypoxic storage bag comprises a hypoxic environment of less than 3500 Pa.

Embodiment 83. A method for preparing stem cells for transplantation in a subject in need thereof comprising: thawing hypoxic stored stem cells; expanding said hypoxic stored stem cells in an hypoxic stem cell expansion system to prepare hypoxic expanded stem cells; and
transplanting said hypoxic expanded stem cells into said subject in need thereof, wherein the hypoxic storage bag comprises a hypoxic environment of less than 3500 Pa.

Embodiment 84. The method of embodiment 83, wherein said stem cell expansion system comprises an outer receptacle substantially impermeable to oxygen, an inner collapsible blood container, an oxygen or oxygen and carbon dioxide sorbent situated between said outer receptacle and said inner collapsible blood container, and at least one inlet/outlet that is substantially impermeable and passing through said outer receptacle and that is in fluid communication with said collapsible container.

Embodiment 85. The method of embodiment 83, wherein said stem cell expansion system comprises a single use bioreactor bag.

Embodiment 86. The method of embodiment 83, wherein said stem cell expansion system comprises 3D cultivation of stem cells in a controlled environment comprising a pO₂ of less than 1400 Pa.

Embodiment 87. The method of embodiment 83, wherein said system comprises a 2D cultivation of stem cells in a controlled environment comprising a pO₂ of less than 1400 Pa.

Embodiment 88. A method for preparing cells for transplantation comprising collecting a blood product containing stem cells into an oxygen absorbing environment comprising an hypoxic collection container, said hypoxic collection container comprising an oxygen (O₂) barrier characterized by an O₂ permeability of less than 0.5 cc of oxygen per square meter per day and an oxygen sorbent; mixing the blood product until the initial partial pressure of oxygen (pO₂) of the blood product is reduced by between 80 and 95% separating leukocytes and stem cells from said blood product comprising applying said blood product to a stem cell binding filter to prepare a leukocyte and stem cells depleted blood product; eluting said stem cells from said filter with an isotonic media; and transferring said stem cells into an hypoxic storage container comprising an inner cell compatible bag comprising a material having an oxygen (O₂) permeability of greater than 25 Barrer and forming hypoxic stored stem cells, wherein said stem cells are not exposed to normoxic conditions for greater than one hour between said collecting and said transferring, wherein said normoxic conditions comprise at least about 21,000 pascal of partial pressure of oxygen.

Embodiment 89. The method of embodiment 88, wherein said separating is performed before said mixing.

### EXAMPLES

### Example 1

### Human Umbilical Cord Blood Collection and Analysis

About 150 mL of HUCB is collected into sterile blood collection bag containing citrate-phosphate-dextrose-adenine 1 (CPDA-1, 21 mL of anticoagulant per 150 mL of HUCB) anticoagulant according to the protocols of the New York Blood Center, New York (USA). Fifty milliliters (50 mL) aliquots are transferred into bags labelled, A, B, and C. Bags A, B, and C are placed on a linear platelet agitator for 3 hours to reduce the percent oxygen saturation in the hemoglobin of the red blood cells to different levels. The HUCB in bag A is the aerobic storage control blood having a saturated oxygen of 80 to 90%. The HUCB in bag B is a hypoxic storage sample having between 10 and 20% SO₂. The HUCB in bag C is a hypoxic storage sample having between 1 and 5% SO₂. The concentrations of the cellular components (leukocytes, platelets and erythrocytes) of HUCB are measured using Sysmex hematology analyzer (Sysmex America, Chicago, IL), the percent oxygen saturation of the hemoglobin of the red blood cells (% SO₂). The partial pressure of oxygen (PO₂ in mmHg) and the partial pressure of carbon dioxide (PCO₂) will be measured with ABL 90 gas analyzer (Radiometer, Brea, CA).

The bags are stored at room temperature for 76 hours after collection and processing. Aliquots (15 mL each) are removed from bags A, B, and C after 24, 36 and 72 hours of storage for analysis of the quality, viability and differentiation of HSCs in the umbilical cord blood.

### Example 2

### Flow Cytometric Analysis of CD34+ Cells in Human Umbilical Cord Blood

The concentrations of CD34+ cells in the pre-filtration HUCB and the recovered cell suspensions are measured using a flow cytometer (FacsCalibur; Becton Dickinson, San Jose, CA, USA) with three-colour direct immunofluorescence system with

Becton Dickinson ProCount tubes (Becton Dickinson). The Becton Dickinson Pro-Count Progenitor Cell Enumeration kit contains CD34+ reagent (Vial ‘A’), control reagent (Vial 13′) and TruCount tubes. The CD34 reagent contains a mixture of the following reagents: a nucleic acid dye that allows for the detection of all nucleated cells including leucocytes and nucleated red blood cells; phycoerythrin (PE)-labelled murine monoclonal CD34 antibody that recognizes the human HSPC present on immature haematopoietic precursor cells and all haematopoietic colony-forming cells in the HUCB; IgG1PE, an isotype control for evaluating non-specific staining or events; and peridinin chlorophyll protein (PerCP)-labelled murine monoclonal antibody CD45 that recognizes human leucocyte antigen that is present in all leucocytes and is weakly expressed or not expressed on haematopoietic cells. Vial ‘B’ of the ProCount kit is the control reagent and it contains a mixture of the nucleic acid dye, PE-labelled IgG1 an isotype control for evaluating non-specific staining or events, as it recognizes only keyhole limpet haemocyanin antigen not expressed on human cells; and CD45-PerCP.

For each HUCB sample, two TrueCount™ tubes are be labelled as “A” for CD34+ (hereinafter “A tubes”) and B for control (hereinafter “B tubes”). Twenty (20) μl of the CD34+ reagent A is transferred into A tubes, and 20 μl of control reagent B into B tubes. Fifty (50) μl of well-mixed HUCB sample (either pre-filtration or recovered from filter) is added to both A and B tubes. The tubes are vortexed gently to mix the sample. All samples are incubated at room temperature (22° C.) in the dark for 15 min. The test samples are combined with a predetermined number of fluorescent beads that served as an internal standard from which the sample volume counted is extrapolated. After 15 min, 450 μl of FACS™ lysing solution is added to both A and B tubes. The tubes are incubated for 30 min in the dark at room temperature. The samples are analyzed according to manufacturer's Lyse/no wash method using the FACSCalibur™ (Becton Dickinson) flow cytometer equipped with an argon laser operated at 15 mW at an excitation wavelength of 488 nm and BD ProCount software 2.0 for automated data acquisition and analysis. The instrument is calibrated daily with labelled beads (Calibrite™, Becton-Dickinson) using FACSComp™ (Becton-Dickinson) software.

The percent recovery of CD34+ cells are calculated using the following formula:

$$Percent recovery ( \% ) = \frac{( Post - filtration concentration of CD 34 / mL + \times Volume )}{( Pre - filtration concentration of CD 34 / mL + \times Volume )} \times 100$$

### Example 2

### Hematopoietic Clonogenic Assay and Cell Viability

Pre-filtration HUCB and cells recovered after filtration of HUCB cells are placed in a colony forming cell (CFC) assay in triplicates at a cell concentration of 3×10⁴ and 1×10⁵ cells per dish. The cells are cultured in a MethoCult™ semisolid matrix. The cultures are incubated at 37° C. 5% CO2 under either normoxic (21% oxygen) or hypoxic conditions (5% oxygen) for 14 days. The colonies are scored based on morphology. In this assay, the progenitor cell populations proliferate and form colonies of recognizable mature cells. Those cells giving rise to colonies are termed CFCs or CFUs and are identified as CFU-erythroid (CFU-E), burst forming unit-erythroid, CFU-granulocyte-macrophage, CFU-granulocyte-erythrocyte-macrophage-megakaryocyte. The data is presented as the number of colonies per 3×10⁵ cells counted for each progenitor cell line. The viability of the cells in HUCB before filtration and in the recovered cells suspensions after filtration are evaluated using trypan blue dye exclusion (StemCell Technologies). A total of 100 cells are counted and the results are expressed in percent viability as the number of live cells (unstained) divided by the total number of cells counted (stained and unstained cells).

### Example 3

### Hypoxic Preparation and Storage of Stem Cells

About 60 to 90 milliliters (mL) of human umbilical cord blood (HUCB) is collected from a donor and into a sterile blood collection bags containing citrate-phosphate-dextrose-adenine 1 (CPDA-1) according to the standard protocol of the New York Blood Center-National Cord Blood Program (NYBC-NCBP). The HUCB is received at the NCBP facility within 24 hours of collection. The concentrations of the cellular components (leukocytes, platelets, and erythrocytes) of HUCB are measured using Sysmex haematology analyzer (Sysmex America, Chicago, IL). The percent oxygen saturation of the hemoglobin of the red blood cells (% SO₂), the partial pressure of oxygen (PO₂ in mmHg) and the partial pressure of carbon dioxide (PCO₂) are measured with an ABL 90 gas analyzer (Radiometer, Brea, CA).

About 20 to 30 mL aliquots of the HUCB are transferred into sterile bags labeled as A (control aerobic storage at 30-90% SO₂ as received), B (hypoxic storage, 10-20% SO₂), and C (hypoxic storage, 0-10% SO₂). The red blood cells present in the HUCB in bags B and C are deoxygenated to between 10 and 20% SO₂ and between 0 and 10% SO₂, respectively. After 24 and 48 hours post deoxygenation to the appropriate level of % SO₂, each sample is tested for haematopoietic clonogenic assay (CFU), viability assay: CD45+ and CD34+ cell viability is assessed by flow cytometry and 7-AAD exclusion)

### Example 4

### Hypoxic Preparation and Storage of Stem Cells

The mean and standard deviation of the data is computed using the statistic program, Prism (Intuitive Software for Sciences, GraphPad, San Diego, CA, USA). Differences in the measured parameters will be analyzed with a two-tailed Student's t-test for both paired and unpaired data with the probability level of less than 0.05 being considered significant. A total of 20 HUCBs are tested for each storage condition on day 0 (before and immediately after 3 hours processing) and, 24, 36 and 76 hours after processing (N=20).

### Example 5

### Hypoxic Collection and Concentration of Stem Cells Using Filtration Technology

Human umbilical cord blood (HUCB) is collected from a patient and directly into a hypoxic collection bag (A) comprising citrate-phosphate dextrose anticoagulant to prevent coagulation of the blood or clumping of the stem cells. The hypoxic collection bag is attached to a stem cell retaining filter attached to a secondary bag for collecting the filtered fluid. The HUCB is depleted of oxygen and filtered with a leukocyte reduction filter for capturing leukocytes and stem cells as provided by FIG. 1. The effluent is collected in a hypoxic storage bag (B). The concentrated stem cells are recovered by flushing the filter containing stem cells with 100 mL of isotonic stem cell culture medium. The stem cells are flushed out in the retrograde position into a hypoxic storage bag (C) containing stem cell maintaining medium, upstream of the filter (FIG. 1).

### Example 6

### Hypoxic Collection and Concentration of Stem Cells Using Centrifugation

Human umbilical cord blood (HUCB), peripheral blood, bone marrow aspirate, or lipoaspirate is collected into a hypoxic collection bag that is connected to two accessory hypoxic storage bags in a hypoxic system. The blood product in the hypoxic collection bag is centrifuged to concentrate the stem cells. The supernatant is removed and the stem cells are resuspended in stem cell maintaining medium. The stem cells are stored in the hypoxic storage bag for up to 3 days, adjusted to the appropriate dose for transplantation while still in the anaerobic storage bag, and then transplanted directly into a patient. Alternatively, the stem cells are diluted with a cryoprotectant and stored in liquid nitrogen at −80° C.

### Example 7

### Hypoxic Expansion of Isolated Progenitor Cells

Stem cells are first collected into a hypoxic storage bag containing the appropriate volume of anticoagulant. The stem cells are isolated and concentrated as described in Example 5 or 6. The HSCs are then transferred from the anaerobic storage bag into a commercially available expansion bags (e.g., GE Healthcare WAVE bioreactor, Terumo Quantum Cell Expansion System) for the expansion of the HSCs under hypoxic storage conditions. The expansion folds range from 3- to 14-fold over 3 to 4 days with a cell viability that is greater than 98%.

The suspension of expanded stem cells are concentrated via centrifugation. The supernatant is removed, and the stem cells are resuspended in stem cell maintaining solution. The stem cell suspension are be adjusted to the appropriate dose for transplantation while still in the anaerobic storage bag. Alternatively, the stem cell suspension is diluted with the appropriate cryoprotectant and then stored in liquid nitrogen (LN) at −80° C.

### Example 8

### Hypoxic Expansion of Isolated Progenitor Cells

The stem cells are collected isolated and concentrated as described in Example 5 or 6. The isolated stem cells are transferred from the hypoxic storage bag into an integrally attached hypoxic bag containing microcarrier beads/scaffold and stem cell expansion medium. The stem cells are expanded for 3 to 4 days. The suspension of stem cells are then concentrated via centrifugation. The supernatant is removed, and the stem cells are resuspended in stem cell maintaining solution. The stem cell suspension is adjusted to the appropriate dose for transplantation while still in the anaerobic storage bag or diluted with the appropriate cryoprotectant and stored at −80° C. or in liquid nitrogen.

### Example 9

### Thawing of Frozen Stem Cell Suspension and Hypoxic Preconditioning

Frozen HSCs, prepared according to Example 7 or 8, are removed from the freezer and exposed to the gas phase for about 15 minutes. The HSCs are then exposed to ambient air for 5 minutes to allow the hypoxic storage bag to regain some elasticity. The hypoxic storage bag is immersed in 37° C. water bath for rapid thawing. After thawing the stem cells are diluted with equal volume of solution containing 2.5% (wt/vol) human albumin and 5% Dextran 40 in isotonic solution. The supernatant is removed via centrifugation and the sedimented stem cells are resuspended slowly in albumin/Dextran solution to the appropriate volume and dose for transplantation.

1. A method for preparing stem cells for transplantation into a patient in need thereof comprising:
collecting a blood product containing stem cells into an oxygen absorbing environment comprising an hypoxic collection container comprising an O₂ barrier characterized by an oxygen (O₂) permeability of less than 0.5 cc of oxygen per square meter per day and an oxygen sorbent;
mixing the blood product until the initial partial pressure of oxygen (pO₂) of the blood product is reduced by between 80 and 95% separating leukocytes and stem cells from said blood product comprising applying said blood product to a filter wherein the stem cells and leukocytes are retained on said filter to prepare a leukocyte and stem cells depleted blood product;
eluting said stem cells from said filter with an isotonic media; and
transferring said stem cells into a hypoxic storage container comprising an O₂ barrier characterized by an oxygen (O₂) permeability of less than 0.5 cc of oxygen per square meter per day and storing said cells to prepare hypoxic stored stem cells.

2. The method of claim 1, further comprising transferring said stem cells to an hypoxic stem cell expansion container and expanding said stem cells to form an expanded stem cell population.

3. The method of claim 2, wherein said transferring said stem cells to said hypoxic stem cell expansion container is prior to said transferring to said hypoxic storage container.

4. The method of claim 1, wherein said mixing, separating, eluting, and transferring are each performed under a pO2 of between 400 and 2000 Pa.

5. The method of claim 1, wherein said mixing is for up to 3 hours.

6. The method of claim 1, wherein said mixing is until the initial partial pressure of oxygen (pO₂) of said blood product is reduced by at least 50%.

7. The method of claim 1, wherein said isotonic media is deoxygenated isotonic media comprising a pO₂ of at least 5333 Pa.

8. The method of claim 1, wherein said transferring comprises directly eluting into said hypoxic storage container.

9. The method of claim 1, wherein said eluting comprises 50 to 200 mL of said isotonic media.

10. The method of claim 1, further comprising equilibrating said isotonic media with oxygen in the air.

11. The method of claim 1, wherein said hypoxic storage container comprises an outer receptacle substantially impermeable to oxygen, a collapsible inner blood container, an oxygen or oxygen and carbon dioxide sorbent situated between said outer receptacle and said inner collapsible blood container, and at least one inlet/outlet that is substantially impermeable and passing through said outer receptacle and that is in hypoxic fluid communication with said collapsible container, wherein said hypoxic storage container comprises less than 1,400 Pa pO₂.

12. The method of claim 1, wherein said blood product is selected from the group consisting of peripheral blood, human umbilical cord blood (HUCB), and bone marrow.

13. A method for preparing stem cells for transfusion comprising:
collecting a blood product comprising stem cells;
separating leukocytes and stem cells from said blood product;
transferring said stem cells into a hypoxic storage container comprising an O₂ barrier characterized by an oxygen (O₂) permeability of less than 0.5 cc of oxygen per square meter per day, and
storing said stem cells in said hypoxic storage container at a partial pressure of oxygen of less than 3500 Pa for a period of time at a temperature of less than 37° C. to form a hypoxic storage bag comprising frozen hypoxic stored stem cells.

14. The method of claim 13, wherein said hypoxic storage container comprises an outer receptacle substantially impermeable to oxygen, a collapsible inner blood container, an oxygen or oxygen and carbon dioxide sorbent situated between said outer receptacle and said collapsible inner blood container, and at least one inlet/outlet that is substantially impermeable and passing through said outer receptacle and that is in fluid communication with said collapsible container, wherein said combination comprises less than 1,400 Pa pO₂.

15. (canceled)

16. (canceled)

17. (canceled)

18. A method of preparing stem cells for transplantation comprising exposing an hypoxic storage bag comprising frozen hypoxic stored stem cells to at least 25° C. to form thawed hypoxic stored stem cells;
centrifuging said thawed hypoxic stored stem cells and removing the supernatant; and
resuspending said hypoxic stored stem cells in a solution comprising 3% dextran 40, wherein the hypoxic storage bag comprises a hypoxic environment of less than 3500 Pa.

19. (canceled)

20. (canceled)

21. The method of claim 18, further comprising expanding said thawed hypoxic stored stem cells.

22. The method of claim 13, wherein said blood product is selected from peripheral blood, human umbilical cord blood (HUCB), and bone marrow.

23. The method of claim 13, wherein said separating comprises centrifugation to prepare concentrated stem cell.

24. The method of claim 23, further comprising removing supernatant from said concentrated stem cells and resuspending said concentrated stem cells in a hypoxic stem cell suspension media.

25. The method of claim 13, wherein said storing is at −80° C. or in liquid nitrogen.
